(19) United States
(12) Patent Application Publication
(10) Pub. No.: US 2010/0037909 A1
(43) Pub. Date: Feb. 18, 2010

(54) SUBSTANCES FOR DYEING KERATINOUS FIBERS

(75) Inventors: Wibke Gross, Dusseldorf (DE); Doris Oberkobush, Dusseldorf (DE); Ralph Nemitz, Juchen (DE); Horst Höffkes, Dusseldorf (DE)

Correspondence Address: CONNOLLY BOVE LODGE & HUTZ LLP, PO BOX 2207, WILMINGTON, DE, 19899-2207, US

(73) Assignee: Henkel AG & Co. KGaA, Dusseldorf (DE)

(21) Appl. No.: 12/440,174

(22) PCT Filed: Aug. 30, 2007
(86) PCT No.: PCT/EP07/59058

§ 371 Date: Mar. 5, 2009

(30) Foreign Application Priority Data
Sep. 5, 2006 (DE) 10 2006 042 075.6

(51) Int. Cl. A61K 8/49; A61Q 5/10

(52) U.S. Cl. 132/208; 8/409

(57) ABSTRACT

Disclosed are substances which contain unsaturated, non-aromatic dialdehydes of formula (Ia) and/or the tautomer (Ib) thereof, wherein R¹, R², and R³ are defined as indicated in claim 1, along with at least one CH-acidic compound of formulas (II) and/or (III), wherein R⁶, R⁷, R⁸, R⁹, R¹⁰, Y, X⁻, Het, and X¹ are defined as indicated in claim 1, in a cosmetic carrier. Said substances color keratinous fibers, especially human hair, in an intensive, colorfast, natural brown shade.

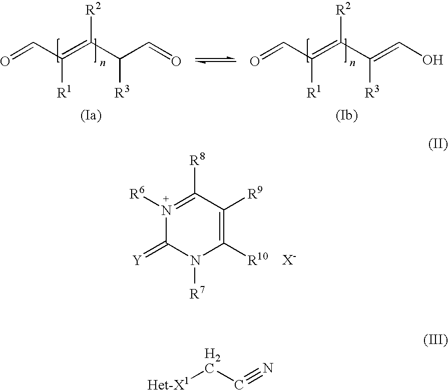

The invention relates to an agent for dyeing keratin-containing fibers, particularly human hair, which comprises specific CH-acidic compounds in combination with unsaturated, non-aromatic dialdehydes, the use of this combination in agents for dyeing keratin-containing fibers, for refreshing the color or nuancing previously dyed keratin-containing fibers as well as a method for dyeing keratin-containing fibers, in particular human hair.

Generally, either substantive dyes or oxidation dyes that result from oxidative coupling of one or more developer components with each other or with one or more coupler components are used for dyeing fibers containing keratin. Coupler components and developer components are also called oxidation dye precursors.

The developer components are normally primary aromatic amines with an additional free or substituted hydroxyl or amino group in the para or ortho position, diaminopyridine derivatives, heterocyclic hydrazones, 4-aminopyrazolone derivatives as well as 2,4,5,6-tetraminopyrimidine and derivatives thereof.

Specific exemplary representatives are p-phenylenediamine, p-toluyienediamine, 2,4,5,6-tetramino pyrimidine, p-amino phenol, N,N-bis(2-hydroxyethyl)-p-phenylenediamine, 2-(2,5-diamino phenyl)-ethanol, 2-(2,5-diamino phenoxy)ethanol, 1-phenyl-3-carboxyamido-4-amino-pyrazolone-5,4-amino-3-methylphenol, 2-aminomethyl-4-amino phenol, 2-hydroxymethyl-4-amino phenol, 2-hydroxy-4,5,6-triamino pyrimidine, 2,4-dihydroxy-5,6-diamino pyrimidine and 2,5,6-triamino-4-hydroxypyrimidine.

m-Phenylenediamine derivatives, naphthols, resorcinol and resorcinol derivatives, pyrazolones, m-amino phenols and substituted pyridine derivatives are generally used as the coupling components.

Particularly suitable coupling substances are α-naphthol, 1,5-dihydroxynaphthalene, 2,7-dihydroxynaphthalene and 1,7-dihydroxynaphthalene, 5-amino-2-methylphenol, m-amino phenol, resorcinol, resorcinol monomethyl ether, m-phenylenediamine, 2,4-diamino phenoxyethanol, 2-amino-4-(2-hydroxyethylamino)-anisole (Lehmann's Blue), 1-phenyl-3-methylpyrazolone-5,2,4-dichloro-3-amino phenol, 1,3-bis(2,4-diamino phenoxy)propane, 2-chlororesorcinol, 4-chlororesorcinol, 2-chloro-6-methyl-3-amino phenol, 2-methylresorcinol, 5-methylresorcinol, 3-amino-6-methoxy-2-methylamino pyridine and 3,5-diamino-2,6-dimethoxypyridine.

In regard to further typical dye components, reference is expressly made to the series “Dermatology”, edited by Ch. Culnan and H. Maibach), Verlag Marcel Dekker Inc., New York, Basel, 1986, volume 7, Ch. Zviak, The Science of Hair Care, chapter 7, (pages 248-250 (substantive dyes), and chapter 8, pages 264-267 (oxidation dyes), as well as the “European Inventory of Cosmetic Raw Materials”, published by the European Union, obtainable in disk form from the Bundesverband Deutscher Industrie-und Handelsunternehmen für Arzneimittel, Reformwaren und Körperpflegemittel e.V., Mannheim.

Indeed, with the oxidation dyes, intensive colorations can be achieved with good fastness characteristics, but the development of the color normally occurs in the presence of oxidizing agents such as e.g. H2O2, which in some cases can result in damage to the fibers. It still proves problematic to prepare oxidative hair colorations in the red tones having adequate fastness characteristics, especially with very good wash fastness and rubbing fastness. Furthermore, some oxidation dye precursors or certain mixtures of oxidation dye precursors sometimes have a sensitizing effect on people with delicate skin. Substantive dyes are applied under more gentle conditions, but their disadvantage is that the colorations often possess only inadequate fastness characteristics.

The object of the present invention is to provide dyes for fibers containing keratin, especially human hair, which in regard to the color depth and fastness characteristics, such as for example light fastness, rubbing fastness and wash fastness as well as fastness to perspiration and cold waving, are qualitatively at least equivalent to the conventional oxidation hair dyes, without, however being necessarily dependent on oxidizing agents such as e.g. H₂O₂. Furthermore, the dyes must have no or only a very slight sensitization potential and in no case may have a mutagenic effect.

Dyes comprising unsaturated, non-aromatic dicarbonyl compounds according to the following Formulas Ia or Ib in combination with the selected CH-acidic compounds of the Formulas (II) or (III), as well as the use of this combination for dyeing fibers containing keratin or for color refreshing or nuancing already dyed fibers containing keratin, are unknown up to now.

CH-acidic 1,2-dihydropyrimidinium derivatives, which are known from the patent application WO-A1-2004/022016, when combined with reactive carbonyl compounds, particularly benzaldehyde derivatives, are suitable for dyeing keratin-containing fibers.

The publication WO-A1-98/47473 relates to unsaturated dicarbonyl compounds that in combination with amines, aromatic hydroxy compounds or CH-acidic compounds represent a colorant.

The palette of purchasable hair dyes comprises, in addition to the light fashion shades, a great variety of natural shades that in particular include a large spectrum of brown nuances. These natural shades are of importance specifically for covering gray hair and regenerating the original hair color.

A brown nuance can be prepared by mixing different light color shades. This requires colorants with yellow components, colorants with a red portion as well as colorants with blue fractions to be used together. This mixing procedure inevitably requires the presence of a great number of colorant components in the dye. As a result of this, application disadvantages often occur, such as for example a different absorption rate of the numerous colorant components on differently damaged parts of the hair, thereby resulting in an irregular coloration. Also, due to dissimilar wash fastness or light fastness characteristics of the various colorants over time, a nuance can be liable to coloration changes that are not wanted by the consumer.

All these disadvantages can be overcome if the desired shade of brown is not the result of additive mixing of yellow, red and blue components, but is rather directly prepared from a combination of two products.

It has now been surprisingly found that by using the inventive CH-acidic compounds of Formulas (II) and/or (III) together with the specifically selected aldehydes of Formula (Ia) or (Ib), only two components are required to achieve very intensive and dark shades of brown on the hair.

A first subject of the invention is a composition for dyeing keratin-containing fibers, particularly human hair, comprising in a cosmetic carrier
as component A at least one unsaturated, non-aromatic dicarbonyl compound according to Formula (Ia) and/or its tautomer (Ib),

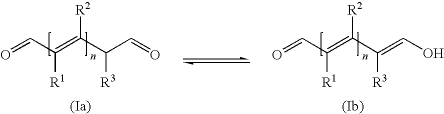

in which
n stands for 1 or 2,
R¹, R² and R³ independently of one another stand for a hydrogen atom, a halogen atom, a (C₁ to C₆) alkyl group, a (C₁ to C₆) alkoxy group, an aryl group, a (C₁ to C₆) alkoxy (C₁ to C₆) alkyl group, wherein for the case that n equals 1, the groups R¹ and R³ or R¹ and R² or R² and R³, can each, together with the remainder of the molecule, form a five-, six- or seven-membered ring that can also be substituted by at least one group selected from a hydrogen atom, a halogen atom, a (C₁ to C₆) alkyl group, a (C₁ to C₆) alkoxy group, an aryl group, a (C₁ to C₆) alkoxy (C₁ to C₆) alkyl group,
   together with
at least one CH-acidic compound as the component B, selected from at least one compound of Formula (II) and/or Formula

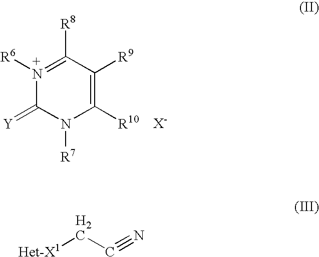

in which
R⁶ and R⁷ independently of one another stand for a linear or cyclic C₁-C₆ alkyl group, a C₂-C₆ alkenyl group, an optionally substituted aryl group, an optionally substituted heteroaryl group, an aryl C₁-C₆ alkyl group, a C₁-C₆ hydroxyalkyl group, a C₂-C₆ polyhydroxyalkyl group, a C₁-C₆ alkoxy C₁-C₆ alkyl group, a R^{I}R^{II}N—(CH₂)ₘ— group, in which R^{I} and R^{II} stand independently of one another for a hydrogen atom, a C₁-C₄ alkyl group, a C₁-C₄ hydroxyalkyl group or an aryl C₁-C₆ alkyl group, wherein R^{I} and R^{II} together with the nitrogen atom can form a 5-, 6- or 7-membered ring and m stands for a number 2, 3, 4, 5 or 6,
R⁸ and R¹⁰ independently of one another stand for a hydrogen atom or a C₁-C₆ alkyl group, wherein at least one of the groups of R⁸ and R¹⁰ means a C₁-C₆ alkyl group,
R⁹ stands for hydrogen, a C₁-C₆ alkyl group or a C₁-C₆ hydroxyalkyl group, a C₂-C₆ polyhydroxy group, a C₁-C₆ alkoxy group, a C₁-C₆ hydroxyalkoxy group, a R^{III}R^{IV}N—(CH₂)_{q}— group, in which R^{III} and R^{IV} stand independently of one another for a hydrogen atom, a C₁-C₆ alkyl group, a C₁-C₆ hydroxyalkyl group or an aryl C₁-C₆ alkyl group and q stands for a number 1, 2, 3, 4, 5 or 6, wherein the group R⁹ together with one of the groups R⁸ or R¹⁰ can form a 5- or 6-membered aromatic ring that can be substituted with a halogen atom, a C₁-C₆ alkyl group, a C₁-C₆ hydroxyalkyl group, a C₂-C₆ polyhydroxyalkyl group, a C₁-C₆ alkoxy group, a C₁-C₆ hydroxyalkoxy group, a nitro group, a hydroxyl group, a group R^{V}R^{VI}N—(CH₂)S—, in which R^{V} and R^{VI} independently of one another stand for a hydrogen atom, a C₁-C₆ alkyl group, a C₁-C₆ hydroxyalkyl group or an aryl C₁-C₆ alkyl group and s stands for a number 0, 1, 2, 3, 4, 5 or 6,
Y stands for an oxygen atom, a sulfur atom or a group NR^{VII}, in which R^{VII} stands for a hydrogen atom, an aryl group, a heteroaryl group, a C₁-C₆ alkyl group or a C₁-C₆ arylalkyl group,
X⁻ stands for a physiologically compatible anion,
Het stands for an optionally substituted heteroaromatic and,
X¹ stands for a direct bond or a carbonyl group.

In the context of the invention, the feature “non-aromatic” means that the carbonyl groups of the dicarbonyl compound according to Formulas (Ia) and (Ib) and to the preferred embodiments of these Formulas (vide infra) are not directly bonded to an aromatic substance.

In general, those compounds are recognized as CH-acidic compounds, which possess a hydrogen atom bonded to an aliphatic carbon atom, wherein the carbon-hydrogen bond is activated due to electron-withdrawing substituents. In principle there are no limits to the choice of the CH-acidic compounds, as long as a compound that is colored to the human eye is obtained after the aldol condensation with a carbonyl compound, in particular with the non-aromatic compound comprised in the inventive agents.

Keratin-containing fibers are understood to mean wool, furs, feathers and particularly human hair. However, the inventive dyes can, in principle, also be used for dyeing other natural fibers, such as e.g. cotton, jute, sisal, linen or silk, modified natural fibers, such as e.g. cellulose regenerate, nitrocellulose, alkyl cellulose or hydroxyalkyl cellulose or acetyl cellulose.

Inventively preferred compounds of the Formula (Ia) or (Ib) are those in which n in said Formulas means the number 1.

It is inventively preferred that when n is equal to 1 in Formula (Ia) or (Ib), then the groups R¹ and R³, together with the remainder of the molecule form a five membered, six membered or seven membered ring that can also be substituted by at least one group selected from a hydrogen atom, a halogen atom, a C₁-C₆ alkyl group, a C₁-C₆ alkoxy group, an aryl group, a C₁-C₆ alkoxy C₁-C₆ alkyl group. Here it is again inventively preferred when the agent comprises at least one compound of the Formulas (Ia-1) and/or its tautomer (Ib-1) as the compound of the Formulas (Ia) or (Ib),

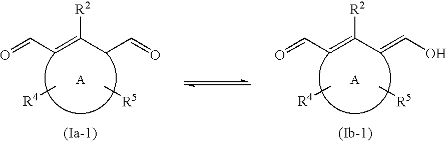

in which
R², R⁴ and R⁵ independently of one another stand for a hydrogen atom, a halogen atom, a C₁-C₆ alkyl group, a C₁-C₆ alkoxy group, an aryl group, a C₁-C₆ alkoxy C₁-C₆ alkyl group and
the ring A is a five membered, six membered or seven membered ring.

The ring A can be carbocyclic or heterocyclic, wherein a carbocyclic ring A is inventively preferred.

The inventive agents quite particularly preferably comprise at least one unsaturated, non-aromatic dicarbonyl compound of the Formula (Ia-2) and/or (Ib-2) and/or (Ia-3) and/or (Ib-3)

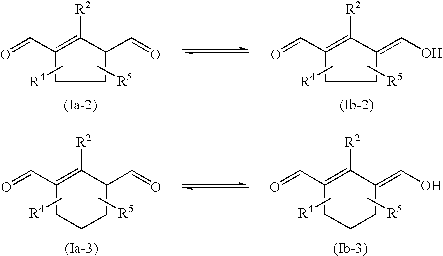

in which
R², R⁴ and R⁵ independently of one another stand for a hydrogen atom, a halogen atom, a C₁-C₆ alkyl group, a C₁-C₆ alkoxy group, an aryl group, a C₁-C₆ alkoxy C₁-C₆ alkyl group.

In accordance with Formulas (Ia), (Ib), (Ia-1), (Ib-1), (Ia-2), (Ib-2), (Ia-3), (Ib-3), it is preferred when R² means a hydrogen atom, a halogen atom (especially chloride or bromide), a C₁-C₆ alkoxy group or a C₁-C₆ alkyl group, more preferably when R² means a hydrogen atom or a halogen atom (especially chloride or bromide).

In accordance with Formulas (Ia-1), (Ib-1), (Ia-2), (Ib-2), (Ia-3), (Ib-3), it is preferred when R⁴ and R⁵ independently of one another mean a hydrogen atom, a hydroxyl group, a C₁-C₆ alkyl group or a C₁-C₆ hydroxyalkyl group. Here it is again preferred when at least one of the groups R⁴ or R⁵ stands for a hydrogen atom and the other means a hydrogen atom, a hydroxyl group, a C₁-C₆ alkyl group or a C₁-C₆ hydroxyalkyl group.

It is inventively preferred that at least one of the following compounds or its tautomer (Ia) is comprised in the inventive composition as the component A corresponding to Formula (Ib):

| | |
|---|---|
| 2-chloro-3-(hydroxymethylene)cyclohex- 1-ene-1-carbaldehyde | 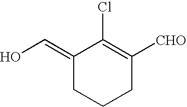 |
| 2-chloro-3-(hydroxymethylene)-5- methylcyclohex-1-ene-1-carbaldehyde | 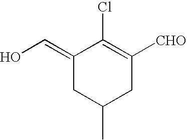 |
| 2-chloro-3-(hydroxymethylene)-6- methylcyclohex-1-ene-1-carbaldehyde | 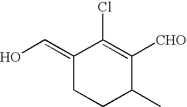 |
| 3-(hydroxymethylene)cyclohex-1-ene-1- carbaldehyde | 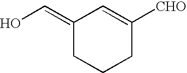 |
| 3-(hydroxymethylene)-5-methylcyclohex-1- ene-1-carbaldehyde | 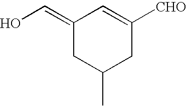 |
| 3-(hydroxymethylene)-6-methylcyclohex-1- ene-1-carbaldehyde | 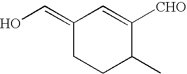 |
| 2-chloro-3-(hydroxymethylene)cyclopent-1- ene-1-carbaldehyde | 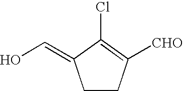 |
| 3-(hydroxymethylene)cyclopent-1-ene-1- carbaldehyde | 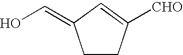 |

Their enamine forms act in the same way as the compounds of Formula II Treatment of the compounds corresponding to Formula II with a base affords the corresponding enamines by deprotonation of the α-carbon atom of the C₁-C₆ alkyl groups R⁸ or R¹⁰. As an example, this deprotonation is illustrated below, wherein for reasons of clarity R⁸ is chosen as the group R—CH₂—. A compound corresponding to the Formula IIa is an example of an inventive enamine form of the 1,2-dihydropyrimidinium derivative.

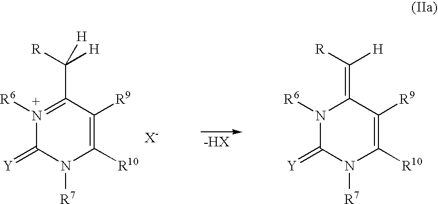

At least one group R⁸ or R¹⁰ of Formula II stands imperatively for a C₁-C₆ alkyl group. This alkyl group preferably carries at least two hydrogen atoms on its α-carbon atom. Particularly preferred alkyl groups are the methyl-, ethyl-, propyl-, n-butyl-, iso-butyl, n-pentyl-, neo-pentyl-, n-hexyl groups. R⁸ and R¹⁰ independently of one another stand quite particularly preferably for hydrogen or a methyl group, wherein at least one of the groups R⁸ or R¹⁰ means a methyl group.

In a preferred embodiment, Y according to Formula II stands for an oxygen atom or a sulfur atom, particularly preferably for an oxygen atom.

The group R⁶ of Formula II is preferably selected from a (C₁-C₆) alkyl group (particularly preferably a methyl group), a C₂-C₆ alkenyl group (especially an allyl group), a hydroxy (C₂ to C₆) alkyl group, particularly a 2-hydroxyethyl group, or an optionally substituted benzyl group.

R⁹ of Formula II preferably stands for a hydrogen atom.

In Formula II at least one of the groups R⁸ and R¹⁰ particularly preferably stands for a methyl group and the other of these groups stands for a hydrogen atom or a methyl group, the group R⁹ for a hydrogen atom, Y for an oxygen atom or a sulfur atom and the groups R⁶ and R⁷ are independently of one another selected from a (C₁-C₆) alkyl group (particularly preferably a methyl group), a C₂-C₆ alkenyl group (especially an allyl group), a hydroxy (C₂ to C₆) alkyl group, particularly a 2-hydroxyethyl group, or an optionally substituted benzyl group

Preferably, the compounds according to Formula II are selected from one or a plurality of compounds of the group of salts with a physiologically compatible counter ion X⁻, which is formed from salts of 1,2-dihydro-1,3,4,6-tetramethyl-2-oxo-pyrimidinium, 1,2-dihydro-1,3-diethyl-4-6-dimethyl-2-oxo-pyrimidinium, 1,2-dihydro-1,3-dipropyl-4-6-dimethyl-2-oxo-pyrimidinium, 1,2-dihydro-1,3-di(2-hydroxyethyl)-4,6-dimethyl-2-oxo-pyrimidinium, 1,2-dihydro-1,3-diphenyl-4-6-dimethyl-2-oxo-pyrimidinium, 1,2-dihydro-1,3,4-trimethyl-2-oxo-pyrimidinium, 1,2-dihydro-1,3-diethyl-4-methyl-2-oxo-pyrimidinium, 1,2-dihydro-1,3-dipropyl-4-methyl-2-oxo-pyrimidinium, 1,2-dihydro-1,3-di(2-hydroxyethyl)-4-methyl-2-oxo-pyrimidinium, 1,2-dihydro-1,3-diphenyl-4-methyl-2-oxo-pyrimidinium, 1-allyl-1,2-dihydro-3,4,6-trimethyl-2-oxo-pyrimidinium, 1,2-dihydro-1-(2-hydroxyethyl)-3,4,6-trimethyl-2-oxo-pyrimidiniums, 1,2-dihydro-1,3,4-6-tetramethyl-2-thioxo-pyrimidinium, 1,2-dihydro-1,3-diethyl-4,6-dimethyl-2-thioxo-pyrimidinium, 1,2-dihydro-1,3-dipropyl-4,6-dimethyl-2-thioxo-pyrimidinium, 1,2-dihydro-1,3-di(2-hydroxyethyl)-4,6-dimethyl-2-thioxo-pyrimidinium, 1,2-dihydro-1,3-diphenyl-4,6-dimethyl-2-thioxo-pyrimidinium, 1,2-dihydro-1,3,4-triphenyl-2-thioxo-pyrimidinium, 1,2-dihydro-1,3-diethyl-4-methyl-2-thioxo-pyrimidinium, 1,2-dihydro-1,3-dipropyl-4-methyl-2-thioxo-pyrimidinium, 1,2-dihydro-1,3-di(2-hydroxyethyl)-4-methyl-2-thioxo-pyrimidinium, 1,2-dihydro-1,3-diphenyl-4-methyl-2-thioxo-pyrimidinium, 1,2-dihydro-3,4-dimethyl-2-oxo-quinazolinium and 1,2-dihydro-3,4-dimethyl-2-thioxo-quinazolinium.

X⁻ in Formula (II) as well as in the above lists preferably stands for halide, benzenesulfonate, p-toluenesulfonate, C₁-C₄ alkanesulfonate, trifluoromethanesulfonate, perchlorate, 0.5 sulfate, hydrogen sulfate, tetrafluoroborate, hexafluorophosphate or tetrachlorozincate. The anions chloride, bromide, iodide, hydrogen sulfate or p-toluenesulfate are particularly preferably employed as X⁻.

The group Het according to Formula (III) preferably stands for the molecule fragment with the Formula (V),

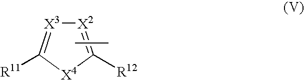

in which
R¹¹ and R¹² stand independently of one another for a hydrogen atom, a hydroxyl group, a halogen atom, a nitro group, a linear or cyclic C₁-C₆ alkyl group, a C₂-C₆ alkenyl group, an optionally substituted aryl group, a cyanomethyl group, a cyanomethyl carbonyl group, an optionally substituted heteroaryl group, an aryl C₁-C₆ alkyl group, a C₁-C₆ hydroxyalkyl group, a C₂-C₆ polyhydroxyalkyl group, a C₁-C₆ alkoxy group, a C₁-C₆ alkoxycarbonyl group, a C₁-C₆ alkoxy C₂-C₆ alkyl group, a C₁-C₆ sulfoalkyl group, a C₁-C₆ carboxyalkyl group, a group R^{VIII}R^{IX}N—(CH₂)ₘ—, in which R^{VIII} and R^{IX} stand independently of one another for a hydrogen atom, a linear or cyclic C₁-C₆ alkyl group, a C₂-C₆ alkenyl group, a C₁-C₆ hydroxyalkyl group or an aryl C₁-C₄ alkyl group, wherein R^{VIII} and R^{IX} together with the nitrogen atom can form a 5-, 6- or 7-membered ring and m stands for a number 0, 1, 2, 3 or 4,
   wherein R¹¹ and/or R¹² can form an optionally substituted aromatic or heteroaromatic 5- or 6-membered ring annulated onto the ring of the residual molecule
X² and X³ stand independently of one another for a nitrogen atom or a CR¹³ group, wherein R¹³ stands for a hydrogen atom, a hydroxyl group, a halogen atom, a nitro group, a linear or cyclic C₁-C₆ alkyl group, a C₂-C₆ alkenyl group, an optionally substituted aryl group, a cyanomethyl group, a cyanomethyl carbonyl group, an optionally substituted heteroaryl group, an aryl C₁-C₆ alkyl group, a C₁-C₆ hydroxyalkyl group, a C₂-C₁₀ polyhydroxyalkyl group, a C₁-C₆ alkoxy group, a C₁-C₆ alkoxycarbonyl group, a C₁-C₆ alkoxy C₂-C₆ alkyl group, a C₁-C₆ sulfoalkyl group, a C₁-C₆ carboxyalkyl group and a group R^{X}R^{XI}N—(CH₂)ₙ—, in which R^{X} and R^{XI} stand independently of one another for a hydrogen atom, a linear or cyclic C₁-C₆ alkyl group, a C₂-C₆ alkenyl group, a C₁-C₆ hydroxyalkyl group or an aryl C₁-C₄ alkyl group, wherein R^{X} and R^{XI} together with the nitrogen atom can form a 5-, 6- or 7-membered ring and n stands for a number 0, 1, 2, 1 or 4, wherein at least one of the substituents X² and X³ can form an optionally substituted aromatic or heteroaromatic 5- or 6-membered ring annulated onto the ring of the residual molecule,
X⁴ stands for an oxygen atom, a sulfur atom, a vinylene group or an N—H group, wherein both the last named groups, independently of one another, can be optionally substituted with a linear or cyclic C₁-C₆ alkyl group, a C₂-C₆ alkenyl group, an optionally substituted aryl group, an optionally substituted heteroaryl group, an aryl C₁-C₆ alkyl group, a C₂-C₆ hydroxyalkyl group, a C₂-C₆ polyhydroxyalkyl group, a C₁-C₆ alkoxy C₂-C₆ alkyl group, a C₁-C₆ sulfoalkyl group, a C₁-C₆ carboxyalkyl group, a group R^{XII}RX^{III}N—(CH₂)ₚ—, in which RXII and R^{XIII} stand independently of one another for a hydrogen atom, a linear or cyclic C₁-C₆ alkyl group, a C₂-C₆ alkenyl group, a C₁-C₆ hydroxyalkyl group or an aryl C₁-C₄ alkyl group, wherein R^{XII} and R^{XIII} together with the nitrogen atom can form a 5-, 6- or 7-membered ring and p stands for a number 0, 1, 2, 3 or 4,
   with the proviso that when X⁴ stands for a vinylene group, at least one of the groups X² or X³ means a nitrogen atom.

The bond between the heterocyclic ring of Formula (V) and the molecule fragment —X¹—CH₂—C≡N with retention of the inventive compound of Formula (III) occurs on the ring of the heterocycle and substitutes a hydrogen atom bonded to this ring. Consequently, it is imperative that the substituents R¹¹, R¹², X², X³ and X⁴ of the Formula (V) are selected such that at least one of these substituents permits the formation of a corresponding bond. Consequently, it is imperative that in Formula (V) at least one of the groups R¹¹ or R¹² forms the bond to the molecule fragment —X¹—CH₂—C≡N, when X⁴ is an oxygen atom or a sulfur atom and X² and X³ mean a nitrogen atom.

The group Het of formula (IV) is particularly preferably derived from the heteroaromatics furan, thiophene, pyrrole, isoxazole, isothiazole, imidazole, oxazole, thiazole, pyridine, pyridazine, pyrimidine, pyrazine, 1,2,3-triazine, 1,2,4-triazine, 1,3,5-triazine, benzopyrrole, benzofuran, benzothiophene, benzimidazole, benzoxazole, indazole, benzoisoxazole, benzoisothiazole, indole, quinoline, isoquinoline, cinnolin, phthalazine, quinazoline, quinoxaline, acridine, benzoquinoline, benzoisoquinoline, benzothiazole, phenazine, benzocinnolin, benzoquinazoline, benzoquinoxaline, phenoxazine, phenothiazine, nephthyridine, phenanthroline, indolizine, quinolizine, carboline, purine, pteridine and coumarine, wherein the above cited heteroaromatics can be substituted with at least one group selected from a halogen atom, a nitro group, a thio group, a thio-(C₁-C₆) alkyl group, a heteroaryl group, an aryl group, a (C₁-C₆) alkyl group, a (C₁-C₆) alkoxy group, a hydroxy group, a (C₂-C₆) hydroxyalkyl group, a (C₂-C₆) polyhydroxyalkyl group, a (C₁-C₆) alkoxyl C₁-C₆ alkyl group, an aryl (C₁-C₆) alkyl group, an amino group, a (C₁-C₆) monoalkylamino group, a (C₁-C₆) dialkylamino group, a dialkylaminoalkyl group —(CH₂)ₙ—NR′R″, in which n is a whole number from 2 and 6 and R′ and R″ independently of one another means a linear or branched alkyl group that can together optionally form a ring.

The compounds according to formula (III) are advantageously selected from the group consisting of 2-(2-furoyl)-acetonitrile, 2-(5-bromo-2-furoyl)-acetonitrile, 2-(5-methyl-2-trifluoromethyl-3-furoyl)-acetonitrile, 3-(2,5-diimethyl-3-furyl)-3-oxopropanitrile, 2-(2-thenoyl)-acetonitrile, 2-(3-thenoyl)-acetonitrile, 2-(5-fluoro-2-thenoyl)-acetonitrile, 2-(5-chloro-2-thenoyl)-acetonitrile, 2-(5-bromo-2-thenoyl)-acetonitrile, 2-(5-methyl-2-thenoyl)-acetonitrile, 2-(2,5-dimethylpyrrol-3-oyl)-acetonitrile, 2-(1,2,5-trimethylpyrrol-3-oyl)-acetonitrile, 1H-benzimidazol-2-yl-acetonitrile, 1H-benzothiazol-2-yl-acetonitrile, 2-(pyrid-2-yl)-acetonitrile, 2,6-bis(cyanomethyl)-pyridine, 2-(indol-3-oyl)-acetonitrile, 2-(2-methyl-indol-3-oyl)-acetonitrile, 8-cyanoacetyl-7-methoxy-4-methylcumarine, 2-(2-isopropyl-5,6-benzoquinolin-4-oyl)-acetonitrile, 2-(2-phenyl-5,6-benzoquinolin-4-oyl)-acetonitrile, 2-(quinoxalin-2-yl)-acetonitrile, 2-(cumaron-2-yl)-acetonitrile, 6,7-dichloro-5-(cyanoacetyl)-2,3-dihydro-1-benzofuran-2-carboxylic acid tert.-butyl ester, 2-(6-hydroxy-4,7-dimethoxy-1-benzofuran-5-oyl)-acetonitrile and 2-(1-phenyl-1,4-dihydrothiochromeno[4,3-c]pyrazol-3-oyl)-acetonitrile. 1H-benzimidazol-2-yl-acetonitrile [2-(cyanomethyl)benzimidazole] is particularly preferred.

Quite particularly preferred inventive agents comprise as the component B at least one CH-acidic compound, selected from
salts of 1,2-dihydro-1,3,4,6-tetramethyl-2-oxopyrimidinium,
salts of 1,2-dihydro-1,3,4-trimethyl-2-oxopyrimidinium,
salts of 1,2-dihydro-1,3,4,6-tetramethyl-2-thioxopyrimidinium,
salts of 1-allyl-1,2-dihydro-3,4,6-trimethyl-2-oxopyrimidinium,
salts of 1,2-dihydro-1-(2-hydroxyethyl)-3,4,6-trimethyl-2-oxopyrimidinium or
2-(cyanomethyl)benzimidazole,
   or mixtures of more than one of these CH-acidic compounds.

In a second embodiment, it can be advantageous for broadening the color spectrum to add to the inventive agents at least one further compound as the component C in addition to at least one compound corresponding to Formula (Ia) or (Ib) as the component A and at least one compound of the component B. The compound of component C is selected from CH-acidic compounds that are different from compounds of the Formulas (II) and (III).

In this context, it has proven inventively advantageous if at least one CH-acidic compound is comprised as the component C, selected from the group consisting of 1,4-dimethylquinolinium, 1-ethyl-4-methyl-quinolinium, 1-ethyl-2-methylquinolinium, 1,2,3,3-tetramethyl-3H-indolium, 2,3-dimethyl-benzothiazolium, 2,3-dimethyl-naphthof 1,2-dithiazolium, 3-ethyl-2-methyl-naphtho[1,2-d]thiazolium, 3-ethyl-2-methyl-benzoxazolium, 1,2,3-trimethylquinoxalinium, 3-ethyl-2-methyl-benzothiazolium, 2,5-dimethyl-3-(2-propenyl)-1,3,4-thiadiazolium, 3-ethyl-2,5-dimethyl-1,3,4-thiadiazolium, 1,2-dimethylquinolinium and 1,3,3-trimethyl-2-methyleneindoline (Fischer base), as well as oxindole, 3-methyl-1-phenyl-pyrazolin-5-one, indan-1,2-dione, indan-1,3-dione, indan-1-one, 2-amino-4-imino-1,3-thiazoline hydrochloride, 4,5-dihydro-4-imino-2-(1-piperidinyl)-thiazole (hydrochloride), 4,5-dihydro-4-imino-2-(4-morpholinyl)-thiazole (hydrochloride), 4,5-dihydro-4-imino-2-(1-pyrrolidinyl)-thiazole (hydrochloride), formed with salts of physiologically compatible anions, in particular p-toluenesulfonates, methanesulfonates, hydrogen sulfates, tetrafluoroborates and halides, such as the chlorides, bromides and iodides.

In a further embodiment, it can be advantageous for broadening the color spectrum to add to the inventive agents at least one further reactive carbonyl compound as the component D in addition to at least one compound corresponding to component A and at least one compound of the component B. Here it is also possible to add at least one additional CH-acidic compound of the component C (vide supra) in addition to a compound corresponding to component A, at least one compound of component B and at least one further reactive carbonyl compound as the component D.

In the context of the invention, reactive carbonyl compounds as the component D possess at least one carbonyl group as the reactive group that reacts with the CH-acidic compound corresponding to component A and or optionally comprised further CH-acidic compounds to form a carbon-carbon bond. Preferred reactive carbonyl compounds are aldehydes and ketones. Moreover, according to the invention, such compounds are also applicable as component D, in which the reactive carbonyl group is derivatized or protected in such a manner that the reactivity of the carbon atom of the derivatized carbonyl group remains towards the CH-acidic compounds of component B. These derivatives are preferably addition compounds
a) of amines and their derivatives, forming imines or oximes as the addition compounds
b) of alcohols, forming acetals or ketals as the addition compounds
c) of water, forming hydrates as the addition product (in this case c), component C is derived from an aldehyde)
   on the carbon atom of the carbonyl group of the reactive carbonyl compound.

Preferred additional reactive carbonyl compounds of the component D are presented below. Here inventive materials are preferred that in addition comprise as the component D at least one reactive carbonyl compound selected from benzaldehyde and its derivatives, cinnamaldehyde and its derivatives, naphthaldehyde and its derivatives, 5-(4-dimethylaminophenyl)penta-2,4-dienal, 5-(4-diethylaminophenyl)penta-2,4-dienal, 5-(4-methoxyphenyl)penta-2,4-dienal, 5-(3,4-dimethoxyphenyl)penta-2,4-dienal, 5-(2,4-dimethoxyphenyl)penta-2,4-dienal, 5-(4-piperidinophenyl)penta-2,4-dienal, 5-(4-morpholinophenyl)penta-2,4-dienal, 5-(4-pyrrolidinophenyl)penta-2,4-dienal, 6-(4-dimethylaminophenyl)hexa-3,5-dien-2-one, 6-(4-diethylaminophenyl)hexa-3,5-dien-2-one, 6-(4-methoxyphenyl)hexa-3,5-dien-2-one, 6-(3,4-dimethoxyphenyl)hexa-3,5-dien-2-one, 6-(2,4-dimethoxyphenyl)hexa-3,5-dien-2-one, 6-(4-piperidinophenyl)hexa-3,5-dien-2-one, 6-(4-morpholinophenyl)hexa-3,5-d ien-2-one, 6-(4-pyrrolidinophenyl)hexa-3,5-dien-2-one, 5-(4-dimethylamino-1-naphthylypenta-3,5-dienal, 9-methyl-3-carbazolaldehyde, 9-ethyl-3-carbazolaldehyde, 3-acetylcarbazole, 3,6-diacetyl-9-ethylcarbazole, 3-acetyl-9-methylcarbazole, 1,4-dimethyl-3-carbazolaldehyde, 1,4,9-trimethyl-3-carbazolaldehyde, 4-formyl-1-methylpyridinium-, 2-formyl-1-methylpyridinium-, 4-formyl-1-ethylpyridinium-, 2-formyl-1-ethylpyridinium-, 4-formyl-1-benzylpyridinium-, 2-formyl-1-benzylpyridinium-, 4-formyl-1,2-dimethylpyridinium-, 4-formyl-1,3-dimethylpyridinium-, 4-formyl-1-methylquinolinium-, 2-formyl-1-methylquinolinium-, 4-acetyl-1-methylpyridinium-, 2-acetyl-1-methylpyridinium-, 4-acetyl-1-methylquinolinium-, 5-formyl-1-methylquinolinium-, 6-formyl-1-methylquinolinium-, 7-formyl-1-methylquinolinium-, 8-formyl-1-methylquinolinium-, 5-formyl-1-ethylquinolinium-, 6-formyl-1-ethylquinolinium-, 7-formyl-1-ethylquinolinium-, 8-formyl-1-ethylquinolinium, 5-formyl-1-benzylquinolinium-, 6-formyl-1-benzylquinolinium-, 7-formyl-1-benzylquinolinium-, 8-formyl-1-benzylquinolinium-, 5-formyl-1-allylquinolinium-, 6-formyl-1-allylquinolinium-, 7-formyl-1-allyiquinolinium- and 8-formyl-1-allylquinolinium-, 5-acetyl-1-methylquinolinium-, 6-acetyl-1-methylquinolinium-, 7-acetyl-1-methylquinolinium-, 8-acetyl-1-methylquinolinium-, 5-acetyl-1-ethylquinolinium-, 6-acetyl-1-ethylquinolinium-, 7-acetyl-1-ethylquinolinium-, 8-acetyl-1-ethylquinolinium-, 5-acetyl-1-benzylquinolinium-, 6-acetyl-1-benzylquinolinium-, 7-acetyl-1-benzylquinolinium-, 8-acetyl-1-benzylquinolinium-, 5-acetyl-1-allylquinolinium-, 6-acetyl-1-allylquinolinium-, 7-acetyl-1-allylquinolinium- and 8-acetyl-1-allylquinolinium-, 9-formyl-10-methylacridinium-, 4-(2′-formylvinyl)-1-methylpyridinium-, 1,3-dimethyl-2-(4′-formylphenyl)benzimidazolium-, 1,3-dimethyl-2-(4′-formylphenyl)imidazolium-, 2-(4′-formylphenyl)-3-methylbenzothiazolium-, 2-(4′-acetylphenyl)-3-methylbenzothiazolium-, 2-(4′-formylphenyl)-3-methylbenzoxazolium-, 2-(5′-formyl-2′-furyl)-3-methylbenzothiazoium-, 2-(5′-formyl-2′-furyl)-3-methylbenzothiazolium-, 2-(5′-formyl-2′-thienyl)-3-methylbenzothiazolium-, 2-(3′-formylphenyl)-3-methylbenzothiazolium-, 2-(4′-formyl-1-naphthyl)-3-methylbenzothiazolium-, 5-chloro-2-(4′-formylphenyl)-3-methylbenzothiazolium-2-(4′-formylphenyl)-3,5-dimethylbenzothiazolium-benzenesulfonate, -p-toluenesulfonate, -methanesulfonate, -perchlorate, -sulfate, -chloride, -bromide, -iodide, -tetrachlorozincate, -methylsulfate-, trifluoromethanesulfonate, -tetrafluoroborate, isatin, 1-methyl-isatin, 1-allyl-isatin, 1-hydroxymethyl-isatin, 5-chloro-isatin, 5-methoxy-isatin, 5-nitroisatin, 6-nitro-isatin, 5-sulfo-isatin, 5-carboxy-isatin, quinisatin, 1-methylquinisatin, as well as any mixtures of the abovementioned compounds.

Benzaldehyde, cinnamaldehyde and naphthaldehyde together with their derivatives, in particular with one or more hydroxyl, alkoxy or amino substituents, are quite particularly preferred as the additional aromatic aldehydes of the component D in the inventive agents. Again, the compounds according to Formula (D-1) are preferred.

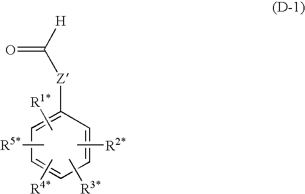

in which
R¹* R²* and R³* independently of each other stand for a hydrogen atom, a halogen atom, a C₁-C₆ alkyl group, a hydroxyl group, a C₁-C₆ alkoxy group, a formyl group, a C₂-C₆ alkenyl group, a C₁-C₁₀ dialkylamino group, a di(C₂-C₆ hydroxyalkyl)amino group, a di(C₁-C₆ alkoxy C₁-C₆ alkyl)amino group, a C₁-C₆ hydroxyalkyloxy group, a sulfonyl group, a carboxyl group, a sulfonic acid group, a sulfonamido group, a sulfonamide group, a carbamoyl group, a C₂-C₆ acyl group or a nitro group,
Z′ stands for a direct bond or a vinylene group,
R⁴* and R⁵* stand for a hydrogen atom or together, with the rest of the molecule, form a 5- or 6-membered aromatic or aliphatic ring.

Here again, inventively preferred agents are those wherein the aromatic aldehyde(s) is/are selected from 4-hydroxy-3-methoxybenzaldehyde, 3,5-dimethoxy-4-hydroxybenzaldehyde, 4-hydroxy-1-naphthaldehyde, 4-hydroxy-2-methoxybenzaldehyde, 3,4-dihydroxy-5-methoxybenzaldehyde, 3,4,5-trihydroxybenzaldehyde, 3,5-dibrom-4-hydroxybenzaldehyde, 4-hydroxy-3-nitrobenzaldehyde, 3-brom-4-hydroxybenzaldehyde, 4-hydroxy-3-methylbenzaldehyde, 3,5-dimethyl-4-hydroxy-benzaldehyde, 5-brom-4-hydroxy-3-methoxybenzaldehyde, 4-diethylamino-2-hydroxybenzaldehyde, 4-dimethylamino-2-methoxybenzaldehyde, coniferyl aldehyde, 2-methoxybenzaldehyde, 3-methoxybenzaldehyde, 4-methoxybenzaldehyde, 2-ethoxybenzaldehyde, 3-ethoxybenzaldehyde, 4-ethoxybenzaldehyde, 4-hydroxy-2,3-dimethoxy-benzaldehyde, 4-hydroxy-2,5-dimethoxy-benzaldehyde, 4-hydroxy-2,6-dimethoxy-benzaldehyde, 4-hydroxy-2-methyl-benzaldehyde, 4-hydroxy-2,3-dimethyl-benzaldehyde, 4-hydroxy-2,5-dimethyl-benzaldehyde, 4-hydroxy-2,6-dimethyl-benzaldehyde, 3,5-diethoxy-4-hydroxy-benzaldehyde, 2,6-diethoxy-4-hydroxy-benzaldehyde, 3-hydroxy-4-methoxy-benzaldehyde, 2-hydroxy-4-methoxy-benzaldehyde, 2-ethoxy-4-hydroxy-benzaldehyde, 3-ethoxy-4-hydroxy-benzaldehyde, 4-ethoxy-2-hydroxy-benzaldehyde, 4-ethoxy-3-hydroxy-benzaldehyde, 2,3-dimethoxybenzaldehyde, 2,4-dimethoxybenzaldehyde, 2,5-dimethoxybenzaldehyde, 2,6-dimethoxybenzaldehyde, 3,4-dimethoxybenzaldehyde, 3,5-dimethoxybenzaldehyde, 2,3,4-trimethoxybenzaldehyde, 2,3,5-trimethoxybenzaldehyde, 2,3,6-trimethoxybenzaldehyde, 2,4,6-trimethoxybenzaldehyde, 2,4,5 trimethoxybenzaldehyde, 2,5,6-trimethoxybenzaldehyde, 2-hydroxybenzaldehyde, 3-hydroxybenzaldehyde, 4-hydroxybenzaldehyde, 2,3-dihydroxybenzaldehyde, 2,4-dihydroxybenzaldehyde, 2,4-dihydroxy-3-methyl-benzaldehyde, 2,4-dihydroxy-5-methyl-benzaldehyde, 2,4-dihydroxy-6-methyl-benzaldehyde, 2,4-dihydroxy-3-methoxy-benzaldehyde, 2,4-dihydroxy-5-methoxy-benzaldehyde, 2,4-dihydroxy-6-methoxy-benzaldehyde, 2,5-dihydroxybenzaldehyde, 2,6-dihydroxybenzaldehyde, 3,4-dihydroxybenzaldehyde, 3,4-dihydroxy-2-methyl-benzaldehyde, 3,4-dihydroxy-5-methyl-benzaldehyde, 3,4-dihydroxy-6-methyl-benzaldehyde, 3,4-dihydroxy-2-methoxy-benzaldehyde, 3,5-dihydroxybenzaldehyde, 2,3,4-trihydroxybenzaldehyde, 2,3,5-trihydroxybenzaldehyde, 2,3,6-trihydroxybenzaldehyde, 2,4,6-trihydroxybenzaldehyde, 2,4,5-trihydroxybenzaldehyde, 2,5,6-trihydroxybenzaldehyde, 4-dimethyl-aminobenzaldehyde, 4-diethylaminobenzaldehyde, 4-dimethylamino-2-hydroxybenzaldehyde, 4-pyrrolidinobenzaldehyde, 4-morpholinobenzaldehyde, 2-morpholinobenzaldehyde, 4-piperidinobenzaldehyde, 3,5-dichloro-4-hydroxybenzaldehyde, 4-hydroxy-3,5-diiodo-benzaldehyde, 3-chloro-4-hydroxybenzaldehyde, 5-chloro-3,4-dihydroxybenzaldehyde, 5-bromo-3,4-dihydroxybenzaldehyde, 3-chloro-4-hydroxy-5-methoxybenzaldehyde, 4-hydroxy-3-iodo-5-methoxybenzaldehyde, 2-methoxy-1-naphthaldehyde, 4-methoxy-1-naphthaldehyde, 2-hydroxy-1-naphthaldehyde, 2,4-dihydroxy-1-naphtaldehyde, 4-hydroxy-3-methoxy-1-naphthaldehyde, 2-hydroxy-4-methoxy-1-naphthaldehyde, 3-hydroxy-4-methoxy-1-naphthaldehyde, 2,4-dimethoxy-1-naphthaldehyde, 3,4-dimethoxy-1-naphthaldehyde, 4-dimethylamino-1-naphthaldehyde, 2-nitrobenzaldehyde, 3-nitrobenzaldehyde, 4-nitrobenzaldehyde, 4-methyl-3-nitrobenzaldehyde, 3-hydroxy-4-nitrobenzaldehyde, 5-hydroxy-2-nitrobenzaldehyde, 2-hydroxy-5-nitrobenzaldehyde, 2-hydroxy-3-nitrobenzaldehyde, 2-fluoro-3-nitrobenzaldehyde, 3-methoxy-2-nitrobenzaldehyde, 4-chloro-3-nitrobenzaldehyde, 2-chloro-6-nitrobenzaldehyde, 5-chloro-2-nitrobenzaldehyde, 4-chloro-2-nitrobenzaldehyde, 2,4-dinitrobenzaldehyde, 2,6-dinitrobenzaldehyde, 2-hydroxy-3-methoxy-5-nitrobenzaldehyde, 4,5-dimethoxy-2-nitrobenzaldehyde, 6-nitropiperonal, 2-nitropiperonal, 5-nitrovanillin, 2,5-dinitrosalicylaldehyde, 5-brom-3-nitrosalicylaldehyde, 4-nitro-1-naphthaldehyde, 2-nitrocinnamaldehyde, 3-nitrocinnamaldehyde, 4-nitrocinnamaldehyde, 4-dimethylaminocinnamaldehyde, 2-dimethylaminobenzaldehyde, 2-chloro-4-dimethylaminobenzaldehyde, 4-dimethylamino-2-methylbenzaldehyde, 4-diethylamino-cinnamaldehyde, 4-dibutylaminobenzaldehyde, 4-diphenylaminobenzaldehyde, 4-(1-imidazolyl)benzaldehyde and piperonal, 5-formyl-2-hydroxybenzoic acid (3-carboxy-4-hydroxybenzaldehyde), 5-formyl-2-hydroxy-3-methoxybenzoic acid (5-carboxyvanillin), 5-formyl-2-hydroxy-3-methylbenzoic acid (3-carboxy-4-hydroxy-5-methylbenzaldehyde), 3-ethoxy-5-formyl-2-hydroxybenzoic acid (3-carboxy-5-ethoxy-4-hydroxybenzaldehyde), 3-allyl-4-hydroyxbenzaldehyde, 3-allyl-4-hydroxy-5-methoxybenzaldehyde, 3-allyl-5-ethoxy-4-hydroxybenzaldehyde, 3-allyl-4-hydroxy-5-methylbenzaldehyde, 3-allyl-5-bromo-4-hydroxybenzaldehyde, 3,5-diallyl-4-hydroxybenzaldehyde, 3-allyl-4,5-dihydroxybenzaldehyde, 3-allyl-4-hydroxy-5-nitrobenzaldehyde, 3-allyl-5-carboxy-4-hydroxybenzaldehyde (3-allyl-5-formyl-2-hydroxybenzoic acid), 5-allyl-4-hydroxyisophthalaldehyde.

In a third embodiment, the dye additionally comprises at least one reaction product (hereafter named reaction product RP) as the substantive dye from a compound of Formula (Ia) or (Ib) and a compound of the component B. These types of reaction products RP can be obtained by e.g. heating both reactants in a neutral to weakly alkaline aqueous medium, wherein the reaction products RP either precipitate out of the solution as a solid or are isolated by evaporating the solution.

For the synthesis of the reaction products RP, molar ratios of the component B to the compound according to Formula (Ia) or (Ib) of about 1:1 to about 2:1 can be reasonable.

The abovementioned compounds of the component A, component B, component C and component D are preferably used in each case in an amount of 0.03 to 65 mmol, in particular, from 1 to 40 mmol, based on 100 g of the total dye. Corresponding inventive agents are preferred, in which the compounds of the component A, the compounds of the component B, and optionally the compounds of the components C and D are each comprised in an amount of 0.03 to 65 mmol, in particular from 1 to 40 mmol, based on 100 g of the total dye.

In addition, the inventive agents can further comprise at least one developer component and optionally at least one coupler component as the oxidation dye precursor.

According to the invention, it may be preferred to use a p-phenylenediamine derivative or one of its physiologically compatible salts as the developer component. Particular preference is given to p-phenylenediamine derivatives of the Formula (E1)

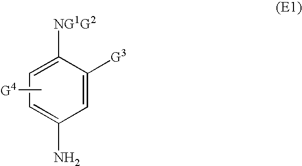

wherein
G¹ stands for a hydrogen atom, a C₁ to C₄ alkyl group, a C₁ to C₄ monohydroxyalkyl group, a C₂ to C₄ polyhydroxyalkyl group, a (C₁ to C₄) alkoxy (C₁ to C₄) alkyl group, a 4′-aminophenyl group or a C₁ to C₄ alkyl group that is substituted by a nitrogen-containing group, a phenyl group or a 4′-aminophenyl group;
G² stands for a hydrogen atom, a C₁- to C₄-alkyl group, a C₁ to C₄ monohydroxyalkyl group, a C₂- to C₄ polyhydroxyalkyl group, a (C₁- to C₄) alkoxy (C₁ to C₄) alkyl group or a C₁ to C₄ alkyl group that is substituted by a nitrogen-containing group;
G³ stands for a hydrogen atom, a halogen atom, such as a chlorine, bromine, iodine or fluorine atom, a C₁ to C₄ alkyl group, a C₁ to C₄ monohydroxyalkyl group, a C₂ to C₄ polyhydroxyalkyl group, a (C₁ to C₄) hydroxyalkyl group, a C₁ to C₄ acetylamino alkoxy group, a C₁ to C₄ mesylamino alkoxy group or a C₁ to C₄ carbamoylamino alkoxy group;
G⁴ stands for a hydrogen atom, a halogen atom or a C₁ to C₄ alkyl group or
when G³ and G⁴ are in the ortho position relative to one another, they can together form a bridging a,ω-alkylenedioxo group, such as, for example, an ethylenedioxy group.

Examples of the C₁ to C₄ alkyl groups specified as substituents in the compounds according to the invention are the methyl, ethyl, propyl, isopropyl and butyl groups. Ethyl and methyl are preferred alkyl groups. Inventively preferred C₁ to C₄ alkoxy groups are a methoxy or ethoxy group, for example. Furthermore, preferred examples of a C₁ to C₄ hydroxyalkyl group that may be mentioned are a hydroxymethyl, a 2-hydroxyethyl, a 3-hydroxypropyl or a 4-hydroxybutyl group. A 2-hydroxyethyl group is particularly preferred. A particularly preferred C₂ to C₄ polyhydroxyalkyl group is the 1,2-dihydroxyethyl group. According to the invention, examples of halogen atoms are F, Cl or Br atoms, Cl atoms being quite particularly preferred. The other terms used are derived according to the invention from the definitions given here. Examples of nitrogen-containing groups of the formula (E1) are, in particular, the amino groups, C₁ to C₄ monoalkylamino groups, C₁; to C₄ dialkylamino groups, C₁ to C₄ trialkylamino groups, C₁ to C₄ monohydroxyalkylamino groups, imidazolinium and ammonium.

Particularly preferred p-phenylenediamines of the formula (E1) are chosen from p-phenylenediamine, p-tolylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)aniline, N,N-bis(β-hydroxyethyl)-p-phenylenediamine, 4-N,N-bis(β-hydroxyethyl)-amino-2-methylaniline, 4-N,N-bis(β-hydroxyethyl)amino-chloroaniline, 2-(β-hydroxyethyl)-p-phenylenediamine, 2-(α,β-dihydroxyethyl)-p-phenylenediamine, 2-fluoro-p-phenylenediamine, 2-isopropyl-p-phenylenediamine, N-(β-hydroxypropyl)-p-phenylenediamine, 2-hydroxymethyl-p-phenylenediamine, N,N-dimethyl-3-methyl-p-phenylenediamine, N,N-(ethyl,β-hydroxyethyl)-p-phenylenediamine, N-(β,γ-dihydroxypropyl)-p-phenylenediamine, N-(4′-aminophenyl)-p-phenylenediamine, N-phenyl-p-phenylenediamine, 2-(β-hydroxyethyloxy)-p-phenylenediamine, 2-(β-acetylaminoethyloxy)-p-phenylenediamine, N-(β-methoxyethyl)-p-phenylenediamine N-(4-amino-3-methylphenyl)-N-[3-(3-)propyl]amine and 5,8-diaminobenzo-1,4-dioxane, and their physiologically compatible salts.

According to the invention, quite particularly preferred p-phenylenediamine derivatives of Formula (E1) are p-phenylenediamine, p-toluyienediamine, 2-(β-hydroxyethyl)-p-phenylenediamine, 2-(α,β-dihydroxyethyl)-p-phenylenediamine and N,N-bis-(β-hydroxyethyl)-p-phenylenediamine.

According to the invention, it may also be preferred to use compounds as the developer component, which comprise at least two aromatic nuclei, that are substituted by amino and/or hydroxyl groups.

Among the binuclear developer components that can be used in the colorant compositions according to the invention, mention may be made in particular, of the compounds which conform to the following formula (E2), together with their physiologically compatible salts:

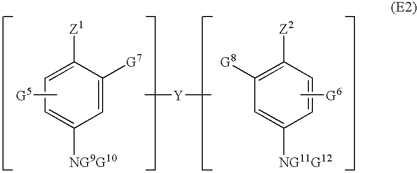

wherein:
Z¹ and Z², independently of one another, stand for a hydroxyl or NH₂ group, which is optionally substituted by a C₁ to C₄ alkyl group, by a C₁ to C₄ hydroxyalkyl group and/or by a bridge Y or which is optionally part of a bridging ring system, the bridge Y stands for an alkylene group containing 1 to 14 carbon atoms, such as, for example, a linear or branched alkylene chain or an alkylene ring, which can be interrupted or terminated by one or more nitrogen-containing groups and/or one or more heteroatoms, such as oxygen, sulfur or nitrogen atoms and may possibly be substituted by one or more hydroxyl or C₁ to C₈ alkoxy groups, or is a direct bond,
G⁵ and G⁶, independently of one another, stand for a hydrogen or halogen atom, a C₁ to C₄ alkyl group, a C₁ to C₄ monohydroxyalkyl group, a C₂ to C₄ polyhydroxyalkyl group, a C₁ to C₄ aminoalkyl group or a direct bond to the bridge Y,
G⁷, G⁸, G⁹, G¹⁰, G¹¹ and G¹², independently of one another, stand for a hydrogen atom, a direct bond to the bridge Y or a C₁ to C₄ alkyl group, with the proviso that the compounds of Formula (E2) comprise only one bridge Y per molecule.

According to the invention, the substituents in Formula (E2) are defined analogously to the above statements.

Preferred binuclear developer components of the Formula (E2) are, in particular: N,N′-bis(β-hydroxyethyl)-N,N′-bis(4′-aminophenyl)-1,3-diaminopropan-2-ol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-diethyl-N,N′-bis(4′-amino-3′-methylphenyl)ethylenediamine, bis(2-hydroxy-5-aminophenyl)methane, 1,3-bis(2,5-diaminophenoxy)propan-2-ol, N,N′-bis(4′-aminophenyl)-1,4-diazacycloheptane, N,N′-bis(2-hydroxy-5-aminobenzyl)piperazine, N-(4′-aminophenyl)-p-phenylenediamine and 1,10-bis(2′,5′-diaminophenyl)-1,4,7,10-tetraoxadecane and their physiologically compatible salts.

Quite particularly preferred binuclear developer components of the Formula (E2) are N,N′-bis(β-hydroxyethyl)-N,N′-bis(4′-aminophenyl)-1,3-diaminopropan-2-ol, bis(2-hydroxy-5-aminophenyl)methane, 1,3-bis(2,5-diaminophenoxy)propan-2-ol, N,N′-bis(4′-aminophenyl)-1,4-diazacycloheptane and 1,10-bis(2,5-diaminophenyl)-1,4,7,10-tetraoxadecane or one of their physiologically compatible salts.

Moreover, according to the invention, it may be preferred to use a p-aminophenol derivative or one of its physiologically compatible salts as the developer component. p-Amino phenol derivatives of the Formula (E3) are particularly preferred.

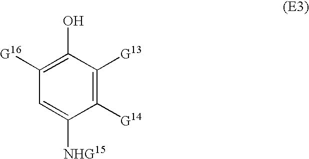

wherein:
G¹³ stands for a hydrogen atom, a halogen atom, a C₁ to C₄ alkyl group, a C₁ to C₄ monohydroxyalkyl group, a C₂ to C₄ polyhydroxyalkyl group, a (C₁ to C₄) alkoxy (C₁ to C₄) alkyl group, a C₁ to C₄ aminoalkyl group, a hydroxy (C₁ to C₄)-alkylamino group, a C₁ to C₄ hydroxyalkoxy group, a C₁ to C₄ hydroxyalkyl (C₁ to C₄) aminoalkyl group or a (di-C₁ to C₄ alkylamino) (C₁ to C₄) alkyl group, and
G¹⁴ stands for a hydrogen or halogen atom, a C₁ to C₄ alkyl group, a C₁ to C₄ monohydroxyalkyl group, a C₂ to C₄ polyhydroxyalkyl group, a (C₁ to C₄) alkoxy (C₁ to C₄) alkyl group, a C₁ to C₄ amino alkyl group or a C₁ to C₄ cyanoalkyl group,
G¹⁵ stands for hydrogen, a C₁ to C₄ alkyl group, a C₁ to C₄ monohydroxyalkyl group, a C₂ to C₄ polyhydroxyalkyl group, a phenyl group or a benzyl group, and
G¹⁶ stands for hydrogen or a halogen atom.

According to the invention, the substituents in Formula (E3) are defined analogously to the above statements.

Preferred p-aminophenols of the Formula (E3) are especially p-aminophenol, N-methyl-p-aminophenol, 4-amino-3-methyl-phenol, 4-amino-3-fluorophenol, 2-hydroxymethylamino-4-aminophenol, 4-amino-3-hydroxymethylphenol, 4-amino-2-(β-hydroxyethoxy)phenol, 4-amino-2-methylphenol, 4-amino-2-hydroxymethylphenol, 4-amino-2-methoxymethylphenol, 4-amino-2-aminomethylphenol, 4-amino-2-(β-hydroxyethyl-aminomethyl)phenol, 4-amino-2-(α,β-dihydroxyethyl)phenol, 4-amino-2-fluorophenol, 4-amino-2-chlorophenol, 4-amino-2,6-dichlorophenol, 4-amino-2-(diethylaminomethyl)phenol together with their physiologically compatible salts.

Quite particularly preferred compounds of the Formula (E3) are p-aminophenol, 4-amino-3-methylphenol, 4-amino-2-aminomethylphenol, 4-amino-2-(α,β-dihydroxyethyl)phenol and 4-amino-2-(diethylaminomethyl)phenol.

Furthermore, the developer component can be selected from β-amino phenol and its derivatives, such as, for example 2-amino-4-methylphenol, 2-amino-5-methylphenol or 2-amino-4-chlorophenol.

In addition, the developer component can be chosen from heterocyclic developer components, such as, for example, the pyridine, pyrimidine, pyrazole, pyrazole-pyrimidine derivatives and their physiologically compatible salts.

Preferred pyridine derivatives are, in particular, the compounds which are described in the patents GB 1 026 978 and GB 1 153 196, such as 2,5-diaminopyridine, 2-(4′-methoxyphenyl)amino-3-aminopyridine, 2,3-diamino-6-methoxypyridine, 2-(β-methoxyethyl)amino-3-amino-6-methoxypyridine and 3,4-diaminopyridine.

Preferred pyrimidine derivatives are, in particular, the compounds which are described in the German patent DE 2 359 399, the Japanese laid-open specification JP 02019576 A2 or in the laid-open specification WO 96/15765, such as 2,4,5,6-tetraminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine and 2,5,6-triaminopyrimidine.

Preferred pyrazole derivatives are, in particular, the compounds which are described in the patents DE 3 843 892, DE 4 133 957 and patent applications WO 94/089691 WO 94/08970, EP 740 931 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert.-butyl-1-methylpyrazole, 4,5-diamino-1-tert.-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole.

Preferred pyrazolo pyrimidine derivatives are, in particular, the derivatives of the pyrazolo[1,5-a]pyrimidine of the following Formula (E4) and its tautomeric forms, provided there is a tautomeric equilibrium:

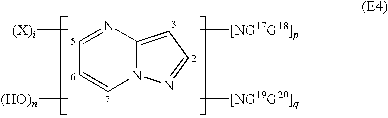

wherein:
G¹⁷, G¹⁸, G¹⁹ and G²⁰ independently of one another stand for a hydrogen atom, a C₁ to C₄ alkyl group, an aryl group, a C₁ to C₄ hydroxyalkyl group, a C₂ to C₄ polyhydroxyalkyl group a (C₁ to C₄) alkoxy (C₁ to C₄) alkyl group, a C₁ to C₄ aminoalkyl group, optionally protected by an acetyl-ureido or a sulfonyl group, a (C₁ to C₄) alkylamino (C₁ to C₄) alkyl group, a di-[(C₁ to C₄) alkyl] (C₁ to C₄) aminoalkyl group, wherein the dialkyl groups optionally form a carbocycle or a heterocycle with 5 or 6 chain members, a C₁ to C₄-hydroxyalkyl- or a di-(C₁ to C₄) [hydroxyalkyl]-(C₁ to C₄) aminoalkyl group,
the X groups independently of one another stand for a hydrogen atom, a C₁ to C₄ alkyl group, an aryl-group, a C₁ to C₄ hydroxyalkyl group, a C₂ to C₄ polyhydroxyalkyl group a (C₁ to C₄) alkoxy (C₁ to C₄) alkyl group, a C₁ to C₄ aminoalkyl group, a (C₁ to C₄) alkylamino (C₁ to C₄) alkyl group, a di-[(C₁ to C₄) alkyl]-(C₁ to C₄) aminoalkyl group, wherein the dialkyl groups optionally form a carbocycle or a heterocycle with 5 or 6 chain members, a C₁ to C₄ hydroxyalkyl- or a di-(C₁ to C₆)—[hydroxyalkyl]-(C₁ to C₄)-aminoalkyl group, an amino group, a C₁ to C₄ alkyl- or a di-(C₁ to C₄ hydroxyalkyl)amino group, a halogen atom, a carboxylic acid group or a sulfonic acid group,
i has the value 0, 1, 2 or 3,
p has the value 0 or 1,
q has the value 0 or 1 and
n has the value 0 or 1,
   with the proviso that
the sum of p+q is not equal to 0,
if p+q is equal to 2, then n has the value 0, and the groups NG¹⁷G¹⁸ and NG¹⁹G²⁰ occupy the positions (2,3); (5,6); (6,7); (3,5) or (3,7);
if p+q is equal to 1, then n has the value 1, and the groups NG¹⁷G¹⁸ (or NG¹⁹G²⁰) and the group OH occupy the positions (2,3); (5,6); (6,7); (3,5) or (3,7);

According to the invention, the substituents in Formula (E4) are defined analogously to the above statements.

If the pyrazolo[1,5-a]pyrimidine of the above formula (E4) comprises a hydroxyl group in one of the positions 2, 5 or 7 of the ring system, there is a tautomeric equilibrium, which is illustrated, for example, in the following scheme:

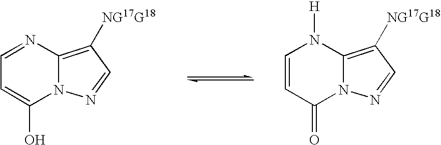

Among the pyrazolo[1,5-a]pyrimidines of the above Formula (E4), mention may be made in particular, of:
Pyrazolo[1,5-a]pyrimidine-3,7-diamine;
2,5-Dimethylpyrazolo[1,5-a]pyrimidine-3,7-diamine;
Pyrazolo[1,5-a]pyrimidine-3,5-diamine;
2,7-Dimethyl-pyrazolo[1,5-a]pyrimidine-3,5-diamine;
3-Aminopyrazolo[1,5-a]pyrimidine-7-ol;
3-Aminopyrazolo[1,5-a]pyrimidine-5-ol;
2-(3-Aminopyrazolo[1,5-a]pyrimidin-7-ylaminoethanol;
2-(7-Aminopyrazolo[1,5-a]pyrimidin-3-ylamino)ethanol;
2-[(3-Aminopyrazolo[1,5-a]pyrimidin-7-yl)-(2-hydroxyethyl)amino]ethanol;
2-[(7-Aminopyrazolo[1,5-a]pyrimidin-3-yl)-(2-hydroxyethyl)amino]ethanol;
5,6-Dimethylpyrazolo[1,5-a]pyrimidine-3,7-diamine;
2,6-Dimethylpyrazolo[1,5-a]pyrimidine-3,7-diamine;
3-Amino-7-dimethylamino-2,5-dimethylpyrazolo[1,5-a]pyrimidine;
   and their physiologically compatible salts and their tautomeric forms if a tautomeric equilibrium exists.

The pyrazolo[1,5-a]pyrimidines of the above formula (E4) can be prepared as described in the literature by cyclization starting from an aminopyrazole or from hydrazine.

In a further preferred embodiment, the dyeing compositions according to the invention comprise at least one coupler component

m-Phenylenediamine derivatives, naphthols, resorcinol and resorcinol derivatives, pyrazolones and m-amino phenol derivatives are generally used as the coupling components. Particularly suitable coupling substances are 1-naphthol, 1,5-, 2,7-, and 1,7-dihydroxynaphthalene, 5-amino-2-methylphenol, m-aminophenol, resorcinol, resorcinol monomethyl ether, m-phenylenediamine, 1-phenyl-3-methylpyrazolone-5,2,4-dichloro-3-aminophenol, 1,3-bis(2′,4′-diaminophenoxy)propane, 2-chlororesorcinol, 4-chlororesorcinol, 2-chloro-6-methyl-3-aminophenol, 2-amino-3-hydroxypyridine, 2-methylresorcinol, 5-methylresorcinol and 2-methyl-4-chloro-5-aminophenol.

Inventively preferred coupling components are
m-aminophenol and its derivatives such as for example 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)-amino-2-methylphenol, 3-(diethylamino)phenol, N-cyclopentyl-3-aminophenol, 1,3-dihydroxy-5-(methylamino)-benzene, 3-ethylamino-4-methylphenol and 2,4-dichloro-3-aminophenol,
o-aminophenol and its derivatives,
m-diaminobenzene and its derivatives such as for example 2,4-diaminophenoxyethanol, 1,3-bis-(2′,4′-diaminophenoxy)propane, 1-methoxy-2-amino-4-(2′-hydroxyethylamino)benzene, 1,3-bis-(2′,4′-diaminophenyl)propane, 2,6-bis-(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-[3-morpholin-4-ylphenyl)amino]ethanol, 3-amino-4-(2-methoxyethoxy)-5-methylphenylamine and 1-amino-3-bis-(2′-hydroxyethyl)-aminobenzene,
o-diaminobenzene and its derivatives such as for example 3,4-diaminobenzoic acid and 2,3-diamino-1-methylbenzene,
di- and trihydroxybenzene derivatives such as, for example, resorcinol, resorcinol monomethyl ether, 2-methyl resorcinol, 5-methyl resorcinol, 2,5-dimethyl resorcinol, 2-chlororesorcinol, 4-chlororesorcinol, pyrogallol and 1,2,4-trihydroxybenzene,
pyridine derivatives such as for example 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine and 3,5-diamino-2,6-dimethoxypyridine,
naphthalene derivatives such as, for example, 1-naphthol, 2-methyl-1-naphthol, 2-hydroxymethyl-1-naphthol, 2-hydroxyethyl-1-naphthol, 1,5-dihydroxynaphthalene, 1,6-dihydroxynaphthalene, 1,7-dihydroxynaphthalene, 1,8-dihydroxynaphthalene, 2,7-dihydroxynaphthalene and 2,3-dihydroxynaphthalene,
morpholine derivatives such as, for example 6-hydroxybenzomorpholine and 6-aminobenzomorpholine,
quinoxaline derivatives such as for example 6-methyl-1,2,3,4-tetrahydroquinoxaline,
pyrazole derivatives such as for example 1-phenyl-3-methylpyrazol-5-one,
indole derivatives such as for example 4-hydroxyindole, 6-hydroxyindole and 7-hydroxyindole,
pyrimidine derivatives such as for example 4,6-diaminopyrimidine, 4-amino-2,6-dihydroxypyrimidine, 2,4-diamino-6-hydroxypyrimidine,
2,4,6-trihydroxypyrimidine, 2-amino-4-methylpyrimidine, 2-amino-4-hydroxy-6-methylpyrimidine and 4,6-dihydroxy-2-methylpyrimidine, or
methylenedioxybenzene derivatives such as for example 1-hydroxy-3,4-methylenedioxybenzene, 1-amino-3,4-methylendioxybenzene and 1-(2′-hydroxyethyl)-amino-3,4-methylenedioxybenzene
   as well as their physiologically compatible salts.

According to the invention, particularly preferred coupler components are 1-naphthol, 1,5-, 2,7- and 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methyl resorcinol, 5-methyl resorcinol, 2,5-dimethyl resorcinol and 2,6-dihydroxy-3,4-dimethylpyridine.

Further, in the context of a fifth embodiment, the precursors of nature-analogous dyes that are used in the inventive agents are preferably those indoles and indolines, which have at least one hydroxyl or amino group, preferably as the substituent on the six-membered ring. These groups can carry further substituents, e.g., in the form of an etherified or esterified hydroxyl group or an alkylated amino group. In a second preferred embodiment, the colorants comprise at least one indole- and/or indoline derivative.

Of particularly good suitability as precursors of nature-analogous hair dyes are derivatives of 5,6-dihydroxyindoline of the Formula (VIa),

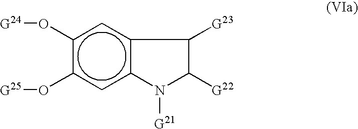

in which, independently of one another
G²¹ stands for hydrogen, a C₁-C₄ alkyl group or a C₁-C₄ hydroxyalkyl group,
G²² stands for hydrogen or a —COOH group, wherein the —COOH group may also be present as the salt with a physiologically compatible cation,
G²³ stands for hydrogen or a C₁-C₄ alkyl group,
G²⁴ stands for hydrogen, a C₁-C₄ alkyl group or a —CO-G²⁶ group, in which G²⁶ stands for a C₁-C₄ alkyl group, and
G²⁵ stands for a group cited under G²⁴,
   as well as physiologically compatible salts of these compounds with an organic or inorganic acid.

Particularly preferred derivatives of indoline are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid and 6-hydroxyindoline, 6-amino indoline and 4-amino indoline.

Within this group, emphasis is placed particularly on N-methyl-5,6-dihydroxyindoline, N-ethyl-5,6-dihydroxyindoline, N-propyl-5,6-dihydroxyindoline, N-butyl-5,6-dihydroxyindoline and, in particular, 5,6-dihydroxyindoline.

In addition, derivatives of 5,6-hydroxyindole of the Formula VIb are exceptionally suitable as precursors of nature-analogous hair dyes,

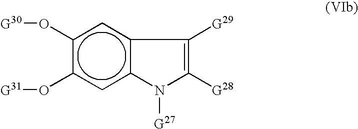

in which, independently of one another
G²⁷ stands for hydrogen, a C₁-C₄ alkyl group or a C₁-C₄ hydroxyalkyl group,
G²⁸ stands for hydrogen or a —COOH group, wherein the —COOH group may also be present as the salt with a physiologically compatible cation,
G²⁹ stands for hydrogen or a C₁-C₄ alkyl group,
G³⁰ stands for hydrogen, a C₁-C₄ alkyl group or a —CO-G³² group, in which G³² stands for a C₁-C₄ alkyl group, and
G³¹ stands for one of the groups cited for G³⁰,
and physiologically compatible salts of these compounds with an organic or inorganic acid.

Particularly preferred derivatives of indole are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-amino indole and 4-amino indole.

Within this group, emphasis is given to N-methyl-5,6-dihydroxyindole, N-ethyl-5,6-dihydroxyindole, N-propyl-5,6-dihydroxyindole, N-butyl-5,6-dihydroxyindole and, in particular, 5,6-dihydroxyindole.

The indoline- and indole derivatives can be employed in the inventive dyes both as free bases and also in the form of their physiologically compatible salts of inorganic or organic acids, e.g. the hydrochlorides, the sulfates and hydrobromides. The indole- or indoline derivatives are comprised in these, usually in amounts of 0.05-10% by weight, preferably 0.2-5% by weight.

The presence of oxidizing agents, e.g. H₂O₂, can be dispensed with, particularly when the inventive agent does not comprise any oxidation dye precursors. If the inventive agent comprises air-oxidizable oxidation dye precursors or indole- or indoline derivatives, then in this case there is no problem with dispensing with oxidizing agents. However, in certain cases hydrogen peroxide or other oxidizing agents can be added to the inventive agent in order to produce nuances that are lighter than the keratinic fibers being dyed. Generally, oxidizing agents are employed in an amount of 0.01 to 6 wt. %, based on the application solution. A preferred oxidizing agent for human hair is H₂O₂. Mixtures of a plurality of oxidizing agents, such as for example a combination of hydrogen peroxide and peroxydisulfates of the alkali metals and alkaline earth metals or sources of iodine ions, such as e.g. alkali metal iodides and hydrogen peroxide or the abovementioned peroxydisulfates, can also be used. In summary, inventive agents are also preferred which additionally comprise oxidizing agents, especially H₂O₂, in a quantity of 0.01 to 6 wt. %, based on the application solution.

According to the invention, the oxidizing agent or the combination of oxidizing agents together with oxidation catalysts can be used in the hair colorant. Oxidation catalysts are for example metal salts, metal chelate complexes or metal oxides, which permit an easy change between two oxidation states of the metal ions. Examples are salts, chelate complexes or oxides of iron, ruthenium, manganese and copper. Enzymes illustrate further possible oxidation catalysts. Suitable enzymes are, for example, peroxidases, which can considerably enhance the effect of small amounts of hydrogen peroxide. Those enzymes that directly oxidize the oxidation dye precursors with the help of atmospheric oxygen are further inventively suitable, such as, for example, the laccases, or those which produce small amounts of hydrogen peroxide in situ and in so doing biocatalytically activate the oxidation of the dye precursors. Particularly suitable catalysts for the oxidation of the dye precursors are the so-called 2-electron oxidoreductases in combination with the substrates specific therefore, e.g.
pyranose-oxidase and e.g. D-glucose or galactose,
glucose-oxidase and D-glucose,
glycerine-oxidase and glycerine,
pyruvate-oxidase and pyruvic acid or its salts,
alcohol-oxidase and alcohol (MeOH, EtOH),
lactate-oxidase and lactic acid or its salts,
tyrosinase-oxidase and tyrosine,
uricase and uric acid or its salts,
choline oxidase and choline,
amino acid oxidase and amino acids.

In another embodiment, in order to further modify the color nuances, the inventive colorants comprise, in addition to the inventively comprised compounds, further conventional substantive dyes, such as nitrophenylenediamine, nitroaminophenols, azo dyes, anthraquinones or indophenols. Preferred substantive dyestuffs are the compounds with the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, Basic Blue 6, C.I.-No. 51,175; Basic Blue 7, C.I.-No. 42,595; Basic Blue 9, C.I.-No. 52,015; Basic Blue 26, C.I.-No. 44,045; Basic Blue 41, C.I.-No. 11,154; Basic Blue 99, C.I.-No. 56,059; Basic Brown 4, C.I.-No. 21,010; Basic Brown 16, C.I.-No. 12,250; Basic Brown 17, C.I.-No. 12,251; Basic Green 1, C.I.-No. 42,040; Basic Orange 31; Basic Red 2, C.I.-No. 50,240; Basic Brown 22, C.I.-No. 11,055; Basic Red 46; Basic Red 51; Basic Red 76, C.I.-No. 12,245; Basic Violet 1, C.I.-No. 42,535; Basic Violet 2; Basic Violet 3, C.I.-No. 42,555; Basic Violet 10, C.I.-No. 45,170; Basic Violet 14, C.I.-No. 42,510; Basic Yellow 57, C.I.-No. 12,719; Basic Yellow 87 and/or the anionic (acidic) dyes, and/or the non-ionic dyes, preferably Acid Black 1, C.I.-No. 20,470; Acid Black 52; Acid Blue 7; Acid Blue 9, C.I.-No. 42,090; Basic Blue 74, C.I.-No. 73,015, Acid Red 18, C.I.-No. 16,255; Acid Red 23; Acid Red 27, C.I.-No. 16,185; Acid Red 33; Acid Red 52; Acid Red 87, C.I.-No. 45,380; Acid Red 92, C.I.-No. 45,410; Acid Orange 3; Acid Orange 7; Acid Violet 43, C.I.-No. 60,730; Acid Yellow 1, C.I.-No. 10,316; Acid Yellow 10; Acid Yellow 23, C.I.-No. 19,140; Acid Yellow 3, C.I.-No. 47,005; Acid Yellow 36; D& C Brown No. 1, C.I.-No. 20,170 (Acid Orange 24); D&C Green No. 5, C.I.-No. 61,570 (Acid Green G); D&C Orange No. 4, C.I.-No. 15,510 (Acid Orange II); D&C Orange No. 10, C.I.-No. 45,425:1 (Solvent Red 73); D&C Orange No. 11, C.I.-No. 45,425 (Acid Red 95); D&C Red No. 21, C.I.-No. 45,380:2 (Solvent Red 43); D&C Red No. 27, C.I.-No. 45,410:1 (Solvent Red 48); D&C Red No. 33, C.I.-No. 17,200 (Acid Red 2A, Acid Red B); D&C Yellow No. 7 C.I.-No. 45,350:1 (Solvent Yellow 94); D&C Yellow No. 8, C.I.-No. 45,350 (Acid Yellow 73); FD& C Red No. 4, C.I.-No. 14,700 (Food Red 4); FD&C Yellow No. 6, C.I.-No. 15,985 (Food Yellow 3); Food Green 3; Pigment Red 57-1; Disperse Black 9; Disperse Blue 1; Disperse Blue 3; Disperse Violet 1; Disperse Violet 4; HC Orange 1; HC Red 1; HC Red 3; HC Red 13; HC Yellow 2; HC Yellow 4 known compounds as well as 1,4-diamino-2-nitrobenzene, 2-amino-4-nitrophenol, 1,4-bis-(β-hydroxyethyl)amino-2-nitrobenzene, 3-nitro-4-(β-hydroxyethyl)-aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-1-hydroxy-4-nitrobenzene.

Preferred agents according to the invention additionally comprise at least one substantive dye, preferably in a quantity of 0.01 to 20 wt. %, based on the total dye.

In addition, the inventive agents can preferably comprise a cationic substantive dye. Particular preference is given here to
(a) cationic triphenylmethane dyes, such as, for example, Basic Blue 7, Basic Blue 26, Basic Violet 2 and Basic Violet 14,
(b) aromatic systems which are substituted by a quaternary nitrogen group, such as, for example, Basic Yellow 57, Basic Red 76, Basic Blue 99, Basic Brown 16 and Basic Brown 17, and
(c) substantive dyes, which comprise a heterocycle that has at least one quaternary nitrogen atom, as are specified, for example, in EP-A2-908 in the claims 6 to 11, which is explicitly incorporated here by reference.

Preferred cationic substantive dyes of group (c) are, in particular, the following compounds:

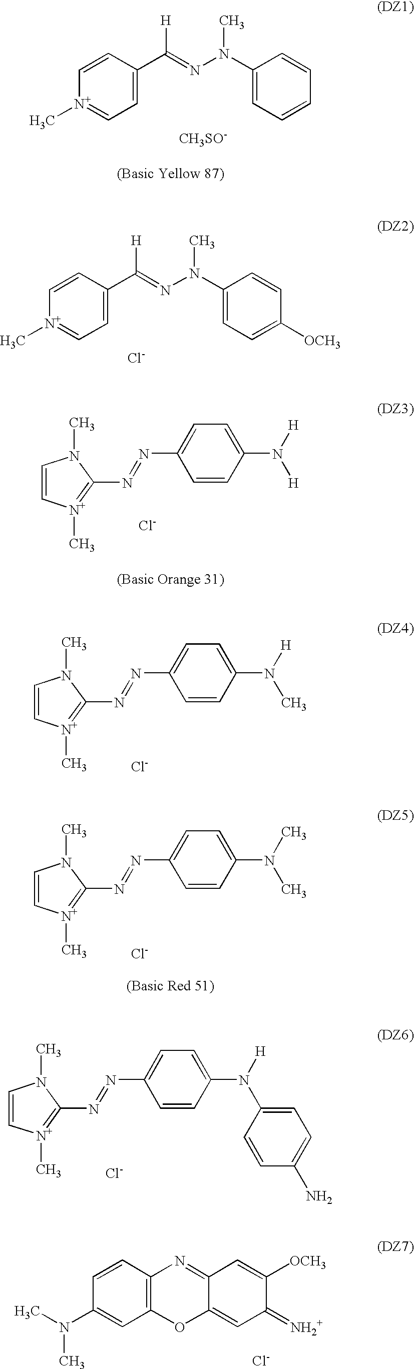

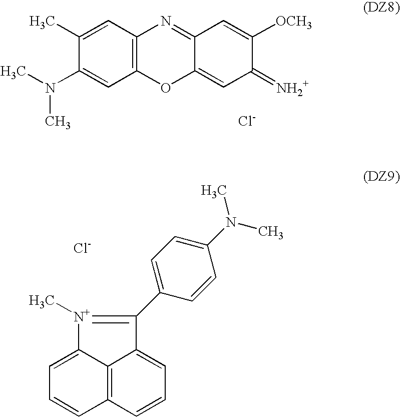

The compounds corresponding to the Formulas (DZ1), (DZ3) and (DZ5) are quite particularly preferred cationic substantive dyes of group (c). According to the invention, the cationic substantive dyes that are commercialized under the trade name Arianor®, are particularly preferred cationic substantive dyes.

The inventive agents according to this embodiment comprise the substantive dyes preferably in a quantity of 0.01 to 20 wt. %, based on the total colorant.

In addition, the inventive preparations can also comprise naturally occurring dyestuffs as are for example, comprised in henna red, henna neutral, henna black, camomile leaves, sandalwood, black tea, alder buckthorn bark, sage, logwood, madder root, cashew, cedar and alkanet root.

It is not required that each of the optionally comprised substantive dyestuffs be pure compounds. In fact, the inventive colorants, due to the manufacturing processes for the individual dyestuffs, may comprise minor quantities of even more components, in so far as they have no detrimental influence on the coloration result or that they must be excluded on other grounds, e.g. toxicological.

To obtain additional and more intensive colorations, the inventive agents can comprise additional color reinforcers. The color reinforcers are preferably selected from at least one compound of the group consisting of piperidine, piperidine-2-carboxylic acid, piperidine-3-carboxylic acid, piperidine-4-carboxylic acid, pyridine, 2-hydroxypyridine, 3-hydroxypyridine, 4-hydroxypyridine, imidazole, 1-methylimidazole, arginine, histidine, pyrrolidine, proline, pyrrolidone, pyrrolidone-5-carboxylic acid, pyrazole, 1,2,4-triazole, piperazidine, their derivatives and their physiologically compatible salts.

Each of the abovementioned color reinforcers can be added in an amount of 0.03 to 10 wt. %, particularly 0.5 to 5 wt. %, each based on 100 g of the ready for use dye.

The pH of the inventive agents can be pH 4 to 12, preferably pH 5 to 10.

The inventive dyes furnish intensive colorations already at physiologically compatible temperatures of below 45° C. In consequence, they are particularly suitable for dyeing human hair. Usually, for use on human hair, the colorants can be incorporated into an aqueous cosmetic carrier. Suitable aqueous cosmetic carriers are, for example, creams, emulsions, gels or also surfactant-containing foaming solutions, such as, for example, shampoos or other preparations that are suitable for use on the keratinic fibers. If necessary, the dyes can also be incorporated into anhydrous carriers. Examples of further suitable and inventively preferred ingredients are given below.

According to the invention, an otherwise customary carrier for agents for dyeing human hair is especially employed as the cosmetic carrier. The inventive dyes can be formulated, apart from the inventive components, with suitably known dyes or comprise typical ingredients for them. Examples of further suitable and inventively preferred ingredients are given below.

The inventive agents preferably comprise the compounds of component A and the compounds of component B in a suitable aqueous, alcoholic or aqueous alcoholic carrier. For the purposes of dyeing the hair, such carriers are, for example, creams, emulsions, gels and also surfactant-containing foaming solutions, such as, for example, shampoos, foam aerosols or other preparations that are suitable for use on the hair. However, it is also conceivable to integrate the dyestuff precursors into a powdered or tablet-shaped formulation.

For the purposes of the present invention, aqueous-alcoholic solutions are understood as meaning aqueous solutions comprising 3 to 70% by weight of a C₁-C₄ alcohol, in particular, ethanol or isopropanol. The compositions according to the invention can additionally comprise further organic solvents, such as, for example, methoxybutanol, benzyl alcohol, ethyl diglycol or 1,2-propylene glycol. Preference here is given to all water-soluble organic solvents.

In many cases the dyes comprise at least one surfactant, wherein, in principal, not only anionic, but also zwitterionic, ampholytic, non-ionic and cationic surfactants are suitable. However, in many cases it has proved advantageous to select the surfactants from among anionic, zwitterionic or non-ionic surfactants, such that inventive agents that additionally comprise anionic, zwitterionic or non-ionic surfactants are preferred.

Suitable anionic surfactants for the inventive preparations are all anionic surface-active materials that are suitable for use on the human body. They are characterized by a water solubilizing anionic group, such as e.g. a carboxylate, sulfate, sulfonate or phosphate group and a lipophilic alkyl group containing about 10 to 22 carbon atoms. In addition, the molecule may comprise glycol or polyglycol ether groups, ester, ether and amide groups as well as hydroxyl groups. Exemplary suitable anionic surfactants are, each in the form of the sodium, potassium and ammonium salts as well as the mono-, di- and trialkanol-ammonium salts with 2 or 3 carbon atoms in the alkanol group,
linear fatty acids containing 10 to 22 carbon atoms (soaps),
ether carboxylic acids of the formula R—O—(CH₂—CH₂O)ₓ—CH₂—COOH, in which R is a linear alkyl group with 10 to 22 carbon atoms and x=0 or 1 to 16,
acyl sarcosides with 10 to 18 carbon atoms in the acyl group,
acyl taurides with 10 to 18 carbon atoms in the acyl group,
acyl isethionates with 10 to 18 carbon atoms in the acyl group,
sulfosuccinic acid mono- and dialkyl esters with 8 to 18 carbon atoms in the alkyl group and sulfosuccinic acid mono-alkyl polyoxyethyl esters with 8 to 18 carbon atoms in the alkyl group and 1 to 6 oxyethylene groups,
linear alkane sulfonates with 12 to 18 carbon atoms, linear alpha-olefin sulfonates with 12 to 18 carbon atoms,
alpha-sulfo fatty acid methyl esters of fatty acids with 12 to 18 carbon atoms,
alkyl sulfates and alkyl polyglycol ether sulfates of formula R—O(CH₂—CH₂O)ₓ—SO₃H, in which R is preferably a linear alkyl group with 10 to 18 carbon atoms and x=0 or 1 to 12,
mixtures of surface-active hydroxy sulfonates according to DE-A-37 25 030,
sulfated hydroxyalkyl polyethylene glycol ethers and/or hydroxyalkylene propylene glycol ethers according to DE-A-37 23 354,
sulfonates of unsaturated fatty acids containing 12 to 24 carbon atoms and 1 to 6 double bonds according to DE-A-39 26 344,
esters of tartaric acid and citric acid with alcohols that represent the addition products of about 2 to 15 molecules of ethylene oxide and/or propylene oxide on fatty alcohols containing 8 to 22 carbon atoms.

Preferred anionic surfactants are alkyl sulfates, alkyl polyglycol ether sulfates and ether carboxylic acids with 10 to 18 carbon atoms in the alkyl group and up to 12 glycol ether groups in the molecule, and especially salts of saturated and particularly unsaturated C₈-C₂₂ carboxylic acids, such as oleic acid, stearic acid, isostearic acid and palmitic acid.

Zwitterionic surfactants are designated as those surface-active compounds that carry at least one quaternary ammonium group and at least one —COO⁽⁻⁾or —SO₃⁽⁻⁾ group in the molecule. Particularly suitable zwitterionic surfactants are the so-called betaines such as the N-alkyl-N,N-dimethylammonium glycinates, for example the cocoalkyl dimethylammonium glycinate, N-acylaminopropyl-N,N-dimethylammonium glycinates, for example the cocoacylaminopropyl dimethylammonium glycinate, and 2-alkyl-3-carboxymethyl-3-hydroxyethyl imidazolines with 8 to 18 carbon atoms in each of the alkyl or acyl groups, as well as cocoacylaminoethyl hydroxyethyl carboxymethyl glycinate. A preferred zwitterionic surfactant is the fatty acid amide derivative, known under the CTFA name Cocamidopropyl betaine.

The ampholytic surfactants are understood to include such surface-active compounds that apart from a C₈₋₁₈ alkyl or acyl group, comprise at least one free amino group and at least one COOH or SO₃H group in the molecule, and are able to form internal salts. Examples of suitable ampholytic surfactants are N-alkyl glycines, N-alkylamino propionic acids, N-alkylamino butyric acids, N-alkylimino dipropionic acids, N-hydroxyethyl-N-alkylamidopropylglycine, N-alkyl taurines, N-alkyl sarcosines, 2-alkylamino propionic acids and alkylamino acetic acids, each with about 8 to 18 carbon atoms in the alkyl group. Particularly preferred ampholytic surfactants are N-cocoalkylamino propionate, the cocoacylaminoethylamino propionate and the C₁₂-C₁₈ acyl sarcosine.

Non-ionic surfactants comprise e.g. a polyol group, a polyalkylene glycol ether group or a combination of polyol and polyglycol ether groups as the hydrophilic group. Exemplary compounds of this type are
Addition products of 2 to 30 moles ethylene oxide and/or 0 to 5 moles propylene oxide to linear fatty alcohols containing 8 to 22 carbon atoms, to fatty acids containing 12 to 22 carbon atoms and to alkyl phenols containing 8 to 15 carbon atoms in the alkyl group,
C₁₂-C₂₂ fatty acid mono- and diesters of addition products of 1 to 30 moles ethylene oxide to glycerine,
C₈₋₂₂ alkyl mono- and oligoglycosides and their ethoxylated analogs,
addition products of 5 to 60 moles ethylene oxide on castor oil and hydrogenated castor oil,
addition products of ethylene oxide on sorbitan fatty acid esters
addition products of ethylene oxide on fatty acid alkanolamides.

Examples of the cationic surfactants that can be used in the inventive hair treatment agents are especially quaternary ammonium compounds. Ammonium halides are preferred, such as alkyl trimethylammonium chlorides, dialkyl dimethylammonium chlorides and trialkyl methylammonium chlorides, e.g. cetyl trimethylammonium chloride, stearyl trimethylammonium chloride, distearyl dimethylammonium chloride, lauryl dimethylammonium chloride, lauryl dimethyl benzylammonium chloride and tricetyl methylammonium chloride. The quaternized protein hydrolyzates illustrate further inventively usable cationic surfactants.

Cationic silicone oils, such as, for example, the commercially available products Q2-7224 (manufacturer: Dow Corning; a stabilized trimethylsilylamodimethicone), Dow Corning 929 emulsion (comprising a hydroxylamino modified silicone, also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, Quaternium-80) are similarly suitable according to the invention.

Alkylamido amines, particularly fatty acid amido amines such as stearylamidopropyldimethylamine, available under the name Tego Amid®S 18, are characterized by a good conditioning action, especially by their good biodegradability.

Quaternary ester compounds, so called “esterquats”, such as methyl hydroxyalkyl dialkoyloxyalkylammonium methosulfates, commercialized under the trade name Stepantex®, also possess very good biodegradability.

An example of a suitable cationic surfactant quaternary sugar derivative is the commercial product Glucquat® 100, “Lauryl methyl gluceth-10 hydroxypropyl dimonium chloride” according to CFTA nomenclature.

For compounds with alkyl groups that are used as surfactants, they may each be pure substances. However, it is normally preferred to start with natural vegetal or animal raw materials for the manufacture of these materials, with the result that mixtures of substances are obtained, which have different alkyl chain lengths that depend on each raw material.

For surfactants, which are represented by the addition products of ethylene oxide and/or propylene oxide to fatty alcohols or derivatives of these addition products, both products with a “normal” homolog distribution as well as those with a narrow homolog distribution may be used. The term “normal” homolog distribution is understood to mean mixtures of homologs obtained from the reaction of fatty alcohols and alkylene oxide using alkali metals, alkali metal hydroxides or alkali metal alcoholates as catalysts. On the other hand, narrow homolog distributions are obtained if e.g. hydrotalcite, alkaline earth metal salts of ether carboxylic acids, alkaline earth metal oxides, hydroxides or alcoholates are used as catalysts. The use of products with a narrow homolog distribution can be preferred.

Additional active substances, auxiliaries and additives are for example
non-ionic polymers, such as, for example, vinyl pyrrolidone/vinyl acrylate copolymers, polyvinyl pyrrolidone and vinyl pyrrolidone/vinyl acetate copolymers and polysiloxanes,
cationic polymers, such as quaternized cellulose ethers, polysiloxanes with quaternary groups, dimethyl diallylammonium chloride polymers, acrylamide-dimethyl diallylammonium chloride copolymers, dimethylaminoethyl methacrylate-vinyl pyrrolidone copolymers quaternized with diethyl sulfate, vinyl pyrrolidone-imidazolinium methochloride copolymers and quaternized polyvinyl alcohol,
zwitterionic and amphoteric polymers, such as, for example, acrylamidopropyl trimethylammonium chloride/acrylate copolymers and octylacrylamide/methyl methacrylate/tert.-butylaminoethyl methacrylate/2-hydroxypropyl methacrylate copolymers,
anionic polymers, such as, for example, polyacrylic acids, crosslinked polyacrylic acids, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert.-butylacrylamide terpolymers,
thickeners like agar-agar, guar gum, alginates, xanthane gum, gum arabica, karaya gum, locust bean flour, linseed gums, dextrans, cellulose derivatives, e.g. methyl cellulose, hydroxyalkyl cellulose and carboxymethyl cellulose, starch fractions and derivatives of amylose, amylopectin and dextrins, clays such as e.g. bentonite or synthetic hydrocolloids such as e.g. polyvinyl alcohol,
structurants such as glucose and maleic acid,
hair conditioning compounds like phospholipids, for example soya lecithin, egg lecithin and cephalin, as well as silicone oils,
protein hydrolyzates, particularly those of elastin, collagen, keratin, milk protein, soya protein and wheat protein, their condensation products with fatty acids as well as quaternized protein hydrolyzates,
perfume oils, dimethyl isosorbitol and cyclodextrins,
solubilizers, such as ethanol, isopropanol, ethylene glycol, propylene glycol, glycerine and diethylene glycol,
anti-dandruff active materials like Piroctone olamine and Zinc omadine,
additional substances for adjusting the pH, such as ammonia, monoethanolamine, basic amino acids and citric acid
active ingredients, such as panthenol, pantothenic acid, allantoin, pyrrolidone carboxylic acids and salts thereof, plant extracts and vitamins,
cholesterol,
UV stabilizers,
thickeners like sugar esters, polyol esters or polyol alkyl ethers,
fats and waxes like spermaceti, beeswax, Montan wax, paraffins, fatty alcohols and fatty acid esters,
fatty acid alkanolamides,
chelating agents like EDTA, NTA and phosphonic acids,
swelling and penetration agents such as glycerol, propylene glycol monoethyl ether, carbonates, hydrogen carbonates, guanidines, ureas, and primary, secondary and tertiary phosphates, imidazoles, tannins,
pyrrole,
opacifiers such as latex,
pearlizers like ethylene glycol mono- and distearate,
blowing agents like propane-butane mixtures, N₂O, dimethyl ether, CO₂ and air, as well as
antioxidants.

The ingredients of the aqueous carrier are added in the usual amounts for the purpose of manufacturing the inventive dyes, e.g. emulsifiers are added in concentrations of 0.5 to 30 wt. % and thickeners in concentrations of 0.1 to 25 wt. % of the total dye.

For the color result it can be advantageous to add ammonium salts or metal salts to the dyes. Suitable metal salts are e.g. formates, carbonates, halides, sulfates, butyrates, valeriates, capronates, acetates, lactates, glycolates, tartrates, citrates, gluconates, propionates, phosphates and phosphonates of alkali metals, such as potassium, sodium or lithium, alkaline earth metals, such as magnesium, calcium, strontium or barium, or of aluminum, manganese, iron, cobalt, copper or zinc, wherein sodium acetate, lithium bromide, calcium bromide, calcium gluconate, zinc chloride, zinc sulfate, magnesium chloride, magnesium sulfate, ammonium carbonate, ammonium chloride and ammonium acetate are preferred. These salts are preferably comprised in an amount of 0.03 to 10 wt. %, particularly 0.5 to 5 wt. %, based on 100 g of the total ready-for-use dye.

The pH of the ready-for-use color preparations is normally between 2 and 11, preferably between 5 and 10.

Another subject matter of the present invention relates to the use of at least one compound corresponding to Formula (Ia) and/or its tautomer (Ib),

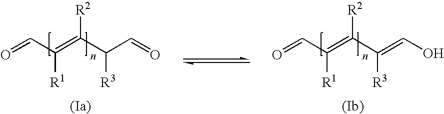

as the component A, in which R¹, R² and R³ are defined as in the first subject matter of the invention,
together with
at least one CH-acidic compound of the Formulas (II) and/or (III) as the component B

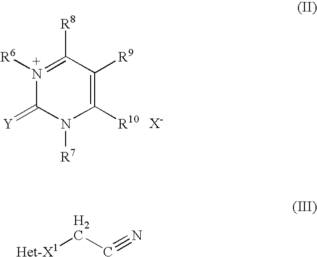

in which R⁶, R⁷, R⁸, R⁹, R¹⁰, Y, X⁻, Het and X¹ are defined as in the first subject matter of the invention,
as the coloring component in hair dyes.

In a preferred embodiment, those compounds corresponding to component A and compounds of the component B, which are selected from the preferred and particularly preferred representatives cited in the first subject matter of the invention, are used as the coloring component in hair dyes.

A third subject matter of the present invention relates to a method for dyeing fibers containing keratin, especially human hair, wherein a dye, comprising
at least one compound corresponding to Formula (Ia) and/or its tautomer (Ib) in a cosmetic carrier as the component A,

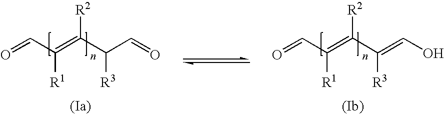

in which R¹, R² and R³ are defined as in the first subject matter of the invention,
   together with
at least one CH-acidic compound of the Formula (II) and/or (III) as the component B,

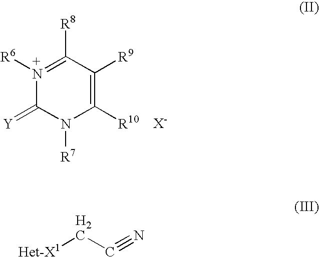

in which R⁶, R⁷, R⁸, R⁹, R¹⁰, Y, X⁻, Het and X¹ are defined as in the first subject matter of the invention,
   is applied onto the keratin-containing fibers, left for a time, usually about 15-30 minutes on the fibers and then rinsed out again or washed out with a shampoo. During the contact time of the agent with the fibers, it can be advantageous to support the dyeing process by supplying heat. The supply of heat can be from a heat source, such as e.g. warm air from a stream of warm air, as also, especially for a hair coloration on living subjects, from the body temperature of the subject. For the latter alternative, the areas being dyed are normally covered with a cap.

Accordingly, the compounds of component A and the compounds of component B, particularly their preferred and particularly preferred representatives cited above, are applied as the coloring components either simultaneously onto the hair or else consecutively, i.e. in a multi-step method, wherein it is irrelevant which component is applied first. The optionally comprised ammonium salts or metal salts can be added to the compounds of the component A or to the compounds of component B. There can be an interval of up to 30 minutes between the addition of the individual components. A pre-treatment of the fibers with the salt solution is also possible.

Before using the inventive agent in the inventive method, it can be desirable to subject the keratin-containing fibers being dyed to a pre-treatment. The time sequence for the required pre-treatment step and the application of the inventive agent does not have to be immediately one after the other, rather the interval between the pre-treatment step and the application of the inventive agent can be up to two weeks at most. There are many methods of pre-treatment. Preferably, the fiber is subjected to

V1 bleaching prior to the application of the inventive agent or
V2 oxidative dyeing prior to the application of the inventive agent.

Corresponding inventive methods, in which the keratin-containing fibers, in the context of a pre-treatment with a hair bleaching agent, were bleached or dyed with an oxidation dye prior to applying a dye according to the invention, are preferred.

In the context of the pre-treatment V1, the keratin-containing fiber is treated with a hair bleaching composition. In addition to an oxidizing agent, such as hydrogen peroxide, the hair bleaching composition preferably comprises at least one inorganic peroxy salt that acts as the oxidation and bleach booster, such as e.g. a peroxydisulfate of sodium, potassium or ammonium. Colorations according to the inventive method acquire a particular brilliance and color depth as a result of the pre-treatment V1.

In the context of the pre-treatment V2, an agent comprising the abovementioned oxidation dye precursors as the developer components and optional coupler components as well as optional abovementioned derivatives of indole or indoline, is applied onto the fiber, and after a contact time, optionally with the addition of abovementioned oxidizing agents on the hair, is left for 5-45 minutes on the keratin fiber. The hair is then rinsed. The existing oxidation colorations can be given a new color nuance by the subsequent application of the inventive agent. By choosing the color nuance of the inventive agent in the same color nuance of the oxidative coloration, then the coloration of the existing oxidation coloration can be refreshed in accordance with the inventive method. It can be seen that the color refreshing or nuancing according to the inventive method is superior in color brilliance and color depth to a color refreshing or nuancing effected solely with conventional substantive dyes.

If in addition to the compounds of the component A and the compounds of the component B, the hair dye comprises hydrogen peroxide as the oxidizing agent or a hydrogen peroxide-containing oxidizing agent mixture, then the pH of the hydrogen peroxide-containing hair dye is preferably in a pH range of pH 7 to pH 11, particularly preferably pH 8 to pH 10. The oxidizing agent can be mixed with the hair dye immediately prior to use and applied to the hair. If the compounds of the component A and the component B are to be applied in a two-step method onto the hair, then the oxidizing agent is used in one of the two steps with the corresponding dyeing component. For this purpose, it can be preferred to package the oxidizing agent with one of the dye components in one container.

The compounds of the component A and the compounds of component B can be stored either in separate containers or together in one container, either in a liquid to pasty preparation (aqueous or anhydrous) or as a solid, for example as a dry powder. If the components are stored together in a liquid preparation then they should be essentially anhydrous and have an acidic pH in order to diminish any reaction of the components. If the components are stored together, then it is preferred to present them as solids, in particular in the form of a preferably multi-layer molded body, e.g. as a tablet. In the case of multi-layered molded bodies, the component A is incorporated into one layer and the component B into another, wherein a further layer preferably lies between the layers as the separation layer. The separation layer is free of compounds of the components A and B. When stored separately, the reactive components are first intimately blended together immediately prior to use. With dry storage, a defined quantity of warm (30° C. to 80° C.) water is normally added before use to prepare a homogeneous mixture.

A fourth subject matter of the invention is the use of
at least one compound corresponding to Formula (Ia) and/or its tautomer (Ib) as the component A,

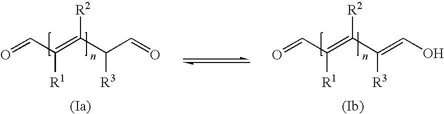

in which R¹, R² and R³ are defined as in the first subject matter of the invention,
together with
at least one CH-acidic compound of the Formula (II) and/or (III) as the component B

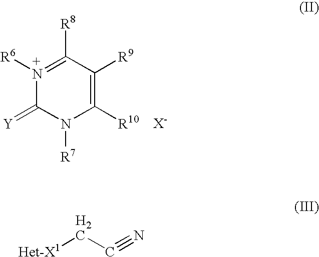

in which R⁶, R⁷, R⁸, R⁹, R¹⁰, Y, X⁻, Het and X¹ are defined as in the first subject matter of the invention,
   for nuancing oxidation colorations of keratin-containing fibers, especially human hair. For use, it is irrelevant whether the nuancing is carried out simultaneously with the oxidative coloration or the oxidative coloration is done before the nuancing.

A fifth subject matter of the invention is the use of
at least one compound corresponding to Formula (Ia) and/or its tautomer (Ib) as the component A,

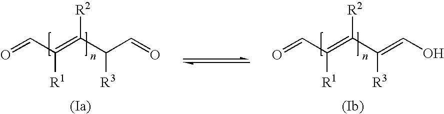

in which R¹, R² and R³ are defined as in the first subject matter of the invention,
   together with
at least one CH-acidic compound of the Formula (II) and/or (III) as the component B,

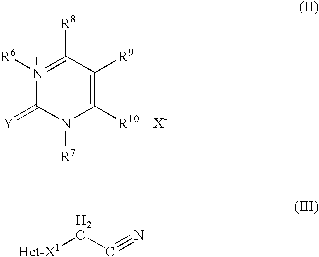

in which R⁶, R⁷, R⁸, R⁹, R¹⁰, Y, X⁻, Het and X¹ are defined as in the first subject matter of the invention,
   for the color refreshment of keratin-containing fibers that were dyed with oxidative dyes.

The colorations of keratin-containing fibers are known to be exposed to environmental influences, such as light, rubbing or washing and can thus lose their brilliance and color depth. In the worst of cases a shift in the nuance of the coloration sometimes occurs. Such changed colorations of keratin-containing fibers can be shifted back, when desired, by a color refreshment to the approximate color state present immediately after the original coloration. According to the invention, a combination of at least one compound of the component A and at least one compound of component B is used for such a color refreshment, such that another subject matter of the present invention is the use of
at least one compound according to Formula (Ia) and/or its tautomer (Ib) as the component A,

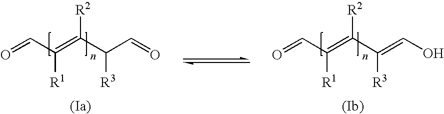

in which R¹, R² and R³ are defined as in the first subject matter of the invention,
   together with
at least one CH-acidic compound of the Formulas (II) and/or (III) as the component B

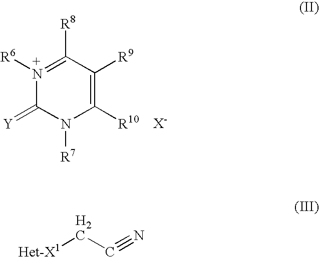

in which R⁶, R⁷, R⁸, R⁹, R¹⁰, Y, X⁻, Het and X¹ are defined as in the first subject matter of the invention,
   for the color refreshment of keratin-containing fibers that were dyed with oxidative dyes.

### EXAMPLES

### Synthetic Examples

### 1.1 Preparation of 2-chloro-3-(hydroxymethylene)cyclohex-1-ene-1-carbaldehyde (A1)

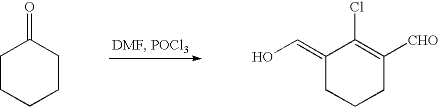

In a 500 ml flask equipped with stirrer, reflux column, internal thermometer and dropping funnel were placed 37.76 g (0.517 mol) dry dimethylformamide (DMF) and cooled in an ice bath. Then, 60.87 g (0.400 mol) phosphorous oxychloride were slowly added drop wise over 45 minutes. The temperature was held between 10 and 15° C. during the drop wise addition. The ice bath was then removed and replaced by a water bath; stirring was continued for 30 minutes. To the thus-prepared Vilsmeier adduct was added drop wise a solution of 10.00 g (0.102 mol) cyclohexanone in 50 ml dried dimethylformamide. After completion of the drop wise addition the water bath was removed and the orange reaction solution was heated for 3 hours at 50 to 60° C.

The orange-red solution was carefully poured onto 300 ml ice, 200 ml water were added and the resulting mixture was stirred. After about 20 minutes a yellow solid began to precipitate out. Stirring was continued over night, the yellow solid was then filtered off and washed several times with water until neutrality. The solid was subsequently dried in the drying oven. Yield=12.9 g (72.9%) melting point=124-126° C.

### 1.2 Preparation of 2-chloro-3-(hydroxymethylene)-5-methylcyclohex-1-ene-1-carbaldehyde (A2)

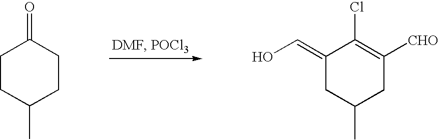

In a 500 ml flask equipped with stirrer, reflux column, internal thermometer and dropping funnel were placed 37.76 g (0.517 mol) dry dimethylformamide (DMF) and cooled in an ice bath. Then, 60.87 g (0.400 mol) phosphorous oxychloride were slowly added drop wise over 45 minutes. The temperature was held between 10 and 15° C. during the drop wise addition. The ice bath was then removed and replaced by a water bath; stirring was continued for 30 minutes. To the thus-prepared Vilsmeier adduct was added drop wise a solution of 11.22 g (0.100 mol) 4-methylcyclohexanone in 55 ml dried dimethylformamide. After completion of the drop wise addition the water bath was removed and the orange reaction solution was heated for 3 hours at 50 to 60° C.

The orange-red solution was carefully poured onto 300 ml ice, 200 ml water were added and the resulting mixture was stirred. After about 20 minutes a yellow solid began to precipitate out. Stirring was continued over night, the solid was then filtered off and washed several times with water until neutrality. The solid was subsequently dried in the drying oven. Yield=13.1 g (70.1%) melting point=140-142° C.

### 1.3 Preparation of 2-chloro-3-(hydroxymethylene)-6-methylcyclohex-1-ene-1-carbaldehyde (A3)

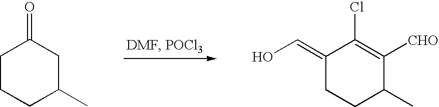

In a 500 ml flask equipped with stirrer, reflux column, internal thermometer and dropping funnel were placed 37.76 g (0.517 mol) dry dimethylformamide (DMF) and cooled in an ice bath. Then, 60.87 g (0.400 mol) phosphorous oxychloride were slowly added drop wise over 45 minutes. The temperature was held between 10 and 15° C. during the drop wise addition. The ice bath was then removed and replaced by a water bath; stirring was continued for 30 minutes. To the thus-prepared Vilsmeier adduct was added drop wise a solution of 11.22 g (0.100 mol) 3-methylcyclohexanone in 55 ml dried dimethylformamide. After completion of the drop wise addition the water bath was removed and the orange reaction solution was heated for 3 hours at 50 to 60° C.

The orange-red solution was carefully poured onto 300 ml ice, 200 ml water were added and the resulting mixture was stirred. After about 20 minutes a yellow solid began to precipitate out. Stirring was continued over night, the yellow-brown solid was then filtered off and washed several times with water until neutrality. The solid was subsequently dried in the drying oven. Yield=11.8 g (63.2%) melting point=135-137° C.

### Dyeing Examples

### 1.0 Manufacture of a dye:

| Aqueous gel formulation for component A | Gel 2 | |
|---|---|---|
| inventive aldehyde (component A) | 10 | mmol |
| Natrosol HR 250 | 2 | g |
| NaOH (50% conc. aqueous solution) | possibly some drops | |
| water, demineralized | ad 100 | g |
| Aqueous gel formulation for component B | Gel 1 | |
| CH-acidic compound (Component B) | 10 | mmol |
| Natrosol HR 250 | 2 | g |
| isopropanol | 10 | g |
| water, demineralized | ad 100 | g |

The inventive aldehyde (component A) was dissolved or suspended in a little water. When needed, the solubility was increased by alkalization with a few drops of 50% sodium hydroxide solution. Subsequently, water was added to make up 98 g and stirring was continued until complete dissolution of the aldehyde (with partial gentle heating to ca. 40° C.). Finally, the Natrosol was added with stirring and the swelling process awaited.

The CH-acidic compound (component B) was firstly dissolved or suspended in a little water with stirring and then made up to 98 g with water. The Natrosol was added with stirring and the swelling process awaited.

### 2.0 Colorations

Both the aqueous gel formulations (Gel 1 and Gel 2) were mixed in the weight ratio 1:1, and then the pH was adjusted with ammonia or tartaric acid to pH 9.

The resulting ready-for use hair dye was applied on a strand of 90% grayed, unpretreated human hair (liquid ratio gel mixture to hair=2:1) and evenly dispersed with an applicette. After a contact time of 30 minutes at 32° C., the strands were rinsed out with lukewarm water and then dried in a stream of warm air. The colorations were visually assessed under a daylight lamp. The result is to be found in Table 1.

TABLE 1
Coloration results

| Component A | Component B | pH | Coloration result |
|---|---|---|---|
| A1 | B1 | 9 | intensively dark brown |
| A1 | B1 | 9 | dark brown |
| A1 | B1 | 9 | medium dark brown |
| A2 | B2 | 9 | intensively dark brown |
| A2 | B2 | 9 | dark brown |
| A2 | B2 | 9 | dark brown |
| A3 | B3 | 9 | light brown |
| A3 | B3 | 9 | medium brown |
| A3 | B3 | 9 | light brown |

A1 2-chloro-3-(hydroxymethylene)cyclohex-1-ene-1-carbaldehyde
A2 2-chloro-3-(hydroxymethylene)-5-methylcyclohex-1-ene-1-carbaldehyde
A3 2-chloro-3-(hydroxymethylene)-6-methylcyclohex-1-ene-1-carbaldehyde
B1 1,2-dihydro-1,3,4,6-tetramethyl-2-oxo-pyrimidinium hydrogen sulfate
B2 1-allyl-1,2-dihydro-3,4,6-trimethyl-2-oxopyrimidinium bromide
B3 1,2-dihydro-1-(2-hydroxyethyl)-3,4,6-trimethyl-2-oxopyrimidinium p-toluene sulfonate

1-20. (canceled)

21. Agent for dyeing keratin-containing fibers, especially human hair, comprising in a cosmetic carrier
as component A at least one unsaturated, non-aromatic dicarbonyl compound according to Formula (Ia) and/or its tautomer (Ib), 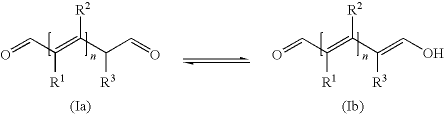
in which
n stands for 1 or 2,
R¹, R² and R³ independently of one another stand for a hydrogen atom, a halogen atom, a (C₁ to C₆) alkyl group, a (C₁ to C₆) alkoxy group, an aryl group, a (C₁ to C₆) alkoxy (C₁ to C₆) alkoxyl group, wherein for the case that n equals 1, the groups R¹ and R³ or R¹ and R² or R² and R³, can each, together with the remainder of the molecule, form a five-, six- or seven-membered ring that can also be substituted by at least one group selected from a hydrogen atom, a halogen atom, a (C₁ to C₆) alkyl group, a (C₁ to C₆) alkoxy group, an aryl group, a (C₁ to C₆) alkoxy (C₁ to C₆) alkyl group,
together with
at least one CH-acidic compound as the component B, selected from at least one compound of Formula (II) and/or Formula (III), 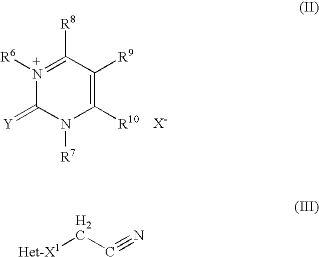
in which
R⁶ and R⁷ independently of one another stand for a linear or cyclic C₁-C₆ alkyl group, a C₂-C₆ alkenyl group, an optionally substituted aryl group, an optionally substituted heteroaryl group, an aryl C₁-C₆ alkyl group, a C₁-C₆ hydroxyalkyl group, a C₂-C₆ polyhydroxyalkyl group, a C₁-C₆ alkoxy C₁-C₆ alkyl group, a R^{I}R^{II}N—(CH₂)ₘ— group, in which R^{I} and R^{II} stand independently of one another for a hydrogen atom, a C₁-C₄ alkyl group, a C₁-C₄ hydroxyalkyl group or an aryl C₁-C₆ alkyl group, wherein R^{I} and R^{II} together with the nitrogen atom can form a 5-, 6- or 7-membered ring and m stands for a number 2, 3, 4, 5 or 6,
R⁸ and R¹⁰ independently of one another stand for a hydrogen atom or a C₁-C₆ alkyl group, wherein at least one of the groups R⁸ and R¹⁰ means a C₁-C₆ alkyl group,
R⁹ stands for hydrogen, a C₁-C₆ alkyl group or a C₁-C₆ hydroxyalkyl group, a C₂-C₆ polyhydroxyalkyl group, a C₁-C₆ alkoxy group, a C₁-C₆ hydroxyalkoxy group, a R^{III}R^{IV}N—(CH₂)_{q}— group, in which R^{III} and R^{IV} stand independently of one another for a hydrogen atom, a C₁-C₆ alkyl group, a C₁-C₆ hydroxyalkyl group or an aryl C₁-C₆ alkyl group and q stands for a number 1, 2, 3, 4, 5 or 6, wherein the group R⁹ together with one of the groups R⁸ or R¹⁰ can form a 5- or 6-membered aromatic ring that can be substituted with a halogen atom, a C₁-C₆ alkyl group, a C₁-C₆ hydroxyalkyl group, a C₂-C₆ polyhydroxyalkyl group, a C₁-C₆ alkoxy group, a C₁-C₆ hydroxyalkoxy group, a nitro group, a hydroxyl group, a group R^{V}R^{VI}N—(CH₂)_{S}—, in which R^{V} and R^{VI} independently of one another stand for a hydrogen atom, a C₁-C₆ alkyl group, a C₁-C₆ hydroxyalkyl group or an aryl C₁-C₆ alkyl group and s stands for a number 0, 1, 2, 3, 4, 5 or 6,
Y stands for an oxygen atom, a sulfur atom or a group NR^{VII}, in which R^{VII} stands for a hydrogen atom, an aryl group, a heteroaryl group, a C₁-C₆ alkyl group or a C₁-C₆ arylalkyl group,
Het stands for an optionally substituted heteroaromatic group and
X¹ stands for a direct bond or a carbonyl group.

22. Agent according to claim 1, wherein n in the Formula (Ia) or (Ib) means the number 1.

23. Agent according to claim 2, wherein the groups R¹ and R³, together with the remainder of the molecule form a five membered, six membered or seven membered ring that can also be substituted by at least one group selected from a hydrogen atom, a halogen atom, a C₁-C₆ alkyl group, a C₁-C₆ alkoxy group, an aryl group, a C₁-C₆ alkoxy C₁-C₆ alkyl group.

24. Agent according to claim 1, comprising at least one unsaturated non-aromatic dicarbonyl compound Formula (Ia-2) and/or (Ib-2) and/or (Ia-3) and/or (Ib-3) as the compound of Formula (Ia) or (Ib) 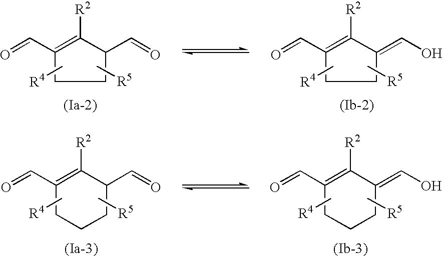
in which
R², R⁴ and R⁵ independently of one another stand for a hydrogen atom, a halogen atom, a C₁-C₆ alkyl group, a C₁-C₆ alkoxy group, an aryl group, a C₁-C₆ alkoxy C₁-C₆ alkyl group.

25. Agent according to claim 1, wherein R² means a hydrogen atom, a halogen atom, a C₁-C₆ alkoxy group or a C₁-C₆ alkyl group.

26. Agent according to claim 3, wherein R⁴ and R⁵ independently of one another mean a hydrogen atom, a hydroxyl group, a (C₁ to C₆) alkyl group or a (C₁ to C₆) hydroxyalkyl group.

27. Agent according to claim 1, wherein at least one of the following compounds or its tautomer of the Formula (Ia) is comprised as the component A corresponding to Formula (Ib):

| | |
|---|---|
| 2-chloro-3-(hydroxy- methylene)cyclohex-1-ene- 1-carbaldehyde | 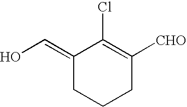 |
| 2-chloro-3-(hydroxymethylene)-5- methylcyclohex-1-ene-1-carbaldehyde | 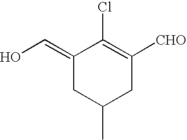 |
| 2-chloro-3-(hydroxymethylene)-6- methylcyclohex-1-ene-1-carbaldehyde | 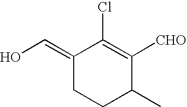 |
| 3-(hydroxymethylene)cyclohex-1-ene-1- carbaldehyde | 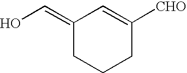 |
| 3-(hydroxy- methylene)-5-methylcyclohex-1-ene- 1-carbaldehyde | 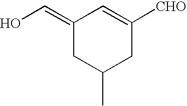 |
| 3-(hydroxymethylene)-6-methylcyclohex- 1-ene-1-carbaldehyde | 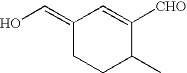 |
| 2-chloro-3-(hydroxy- methylene)cyclopent-1-ene- 1-carbaldehyde | 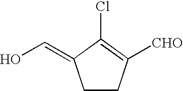 |
| 3-(hydroxymethylene)cyclopent-1-ene-1- carbaldehyde | 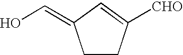 |

28. Agent according to claim 1, wherein as the component B at least one compound is comprised that is selected from the group consisting of:
1,2-dihydro-1,3,4,6-tetramethyl-2-oxo-pyrimidinium, 1,2-dihydro-1,3-diethyl-4,6-dimethyl-2-oxo-pyrimidinium, 1,2-dihydro-1,3-dipropyl-4,6-dimethyl-2-oxo-pyrimidinium, 1,2-dihydro-1,3-di(2-hydroxyethyl)-4,6-dimethyl-2-oxo-pyrimidinium, 1,2-dihydro-1,3-diphenyl-4,6-dimethyl-2-oxo-pyrimidinium, 1,2-dihydro-1,3,4-trimethyl-2-oxo-pyrimidinium, 1,2-dihydro-1,3-diethyl-4-methyl-2-oxo-pyrimidinium, 1,2-dihydro-1,3-dipropyl-4-methyl-2-oxo-pyrimidinium, 1,2-dihydro-1,3-di(2-hydroxyethyl)-4-methyl-2-oxo-pyrimidinium, 1,2-dihydro-1,3-diphenyl-4-methyl-2-oxo-pyrimidinium, 1-alkyl-1,2-dihydro-3,4,6-trimethyl-2-oxo-pyrimidinium, 1,2-dihydro-1-(2-hydroxyethyl)-3,4,6-trimethyl-2-oxo-pyrimidinium, 1,2-dihydro-1,3,4,6-tetramethyl-2-thioxo-pyrimidinium, 1,2-dihydro-1,3-diethyl-4,6-dimethyl-2-thioxo-pyrimidinium, 1,2-dihydro-1,3-dipropyl-4,6-dimethyl-2-thioxo-pyrimidinium, 1,2-dihydro-1,3-di(2-hydroxyethyl)-4,6-dimethyl-2-thioxo-pyrimidinium, 1,2-dihydro-1,3-diphenyl-4,6-dimethyl-2-thioxo-pyrimidinium, 1,2-dihydro-1,3,4-trimethyl-2-thioxo-pyrimidinium, 1,2-dihydro-1,3-diethyl-4-methyl-2-thioxo-pyrimidinium, 1,2-dihydro-1,3-dipropyl-4-methyl-2-thioxo-pyrimidinium, 1,2-dihydro-1,3-di(2-hydroxyethyl)-4-methyl-2-thioxo-pyrimidinium, 1,2-dihydro-1,3-diphenyl-4-methyl-2-thioxo-pyrimidinium, 1,2-dihydro-3,4-dimethyl-2-oxo-quinazolinium, 1,2-dihydro-3,4-dimethyl-2-thioxo-quinazolinium, 2-(2-furoyl)-acetonitrile, 2-(5-bromo-2-furoyl)-acetonitrile, 2-(5-methyl-2-trifluoromethyl-3-furoyl)-acetonitrile, 3-(2,5-dimethyl-3-furyl)-3-oxopropanitrile, 2-(2-thenoyl)-acetonitrile, 2-(3-thenoyl)-acetonitrile, 2-(5-fluoro-2-thenoyl)-acetonitrile, 2-(5-chloro-2-thenoyl)-acetonitrile, 2-(5-bromo-2-thenoyl)-acetonitrile, 2-(5-methyl-2-thenoyl)-acetonitrile, 2-(2,5-dimethylpyrrol-3-oyl)-acetonitrile, 2-(1,2,5-trimethylpyrrol-3-oyl)-acetonitrile, 1H-benzimidazol-2-ylacetonitrile, 1H-benzothiazol-2-ylacetonitrile, 2-(pyrid-2-yl)-acetonitril e, 2,6-bis(cyanomethyl)-pyridine, 2-(indol-3-oyl)-acetonitrile, 2-(2-methylindol-3-oyl)-acetonitrile, 8-cyanoacetyl-7-methoxy-4-methylcoumarin, 2-(2-isopropyl-5,6-benzoquinolin-4-oyl)-acetonitrile, 2-(2-phenyl-5,6-benzoquinolin-4-oyl)-acetonitrile, 2-(quinoxalin-2-yl)-acetonitrile, 2-(coumaron-2-yl)-acetonitrile, 6,7-dichloro-5-(cyanoacetyl)-2,3-dihydro-1-benzofuran-2-carboxylic acid tert.-butyl ester, 2-(6-hydroxy-4,7-dimethoxy-1-benzofuran-5-oyl)-acetonitrile and 2-(1-phenyl-1,4-dihydrothiochromeno[4,3-c]pyrazol-3-oyl)-acetonitrile.

29. Agent according to claim 1, wherein at least one CH-acidic compound is additionally comprised as the component C, selected from the group consisting of 1,4-dimethylquinolinium, 1-ethyl-4-methyl-quinolinium, 1-ethyl-2-methylquinolinium, 1,2,3,3-tetramethyl-3H-indolium, 2,3-dimethyl-benzothiazolium, 2,3-dimethyl-naphtho[1,2-d]thiazolium, 3-ethyl-2-methyl-naphtho[1,2-d]thiazolium, 3-ethyl-2-methyl-benzoxazolium, 1,2,3-trimethylquinoxalinium, 3-ethyl-2-methyl-benzothiazolium, 1,2-dihydro-1,3,4,6-tetramethyl-2-oxo-pyrimidinium, 1,2-dihydro-1,3,4-trimethyl-2-oxo-pyrimidinium, 1,2-dihydro-4,6-dimethyl-1,3-dipropyl-2-oxo-pyrimidinium, 1,2-dihydro-1,3,4,6-tetramethyl-2-thioxo-pyrimidinium, 1,2-dihydro-1,3,4,5,6-pentamethyl-2-oxo-pyrimidinium, 1-allyl-1,2-dihydro-3,4,6-trimethyl-2-oxo-pyrimidinium, 2,5-dimethyl-3-(2-propenyl)-1,3,4-thiadiazolium, 3-ethyl-2,5-dimethyl-1,3,4-thiadiazolium, 1,2-dimethylquinolinium and 1,3,3-trimethyl-2-methyleneindoline (Fischer base), as well as oxindole, 3-methyl-1-phenyl-pyrazolin-5-one, indan-1,2-dione, indan-1,3-dione, indan-1-one, 2-amino-4-imino-1,3-thiazoline hydrochloride, 3-dicyanomethylenindan-1-on, 2-(2-furanoyl)acetonitril, 2-(2-thenoyl)acetonitril, 4,5-dihydro-4-imino-2-(1-piperidinyl)-thiazole (hydrochloride), 4,5-dihydro-4-imino-2-(4-morpholinyl)-thiazole (hydrochloride), 4,5-dihydro-4-imino-2-(1-pyrrolidinyl)-thiazole (hydrochloride), formed with salts of physiologically compatible anions, in particular p-toluenesulfonates, methanesulfonates, hydrogen sulfates, tetrafluoroborates and halides, such as the chlorides, bromides and iodides.

30. Agent according to claim 1, additionally comprising as the component D at least one reactive carbonyl compound that is selected from at least one compound of the group consisting of benzaldehyde and its derivatives, cinnamaldehyde and its derivatives, naphthaldehyde and its derivatives, 5-(4-dimethylaminophenyl)penta-2,4-dienal, 5-(4-diethylaminophenyl)penta-2,4-dienal, 5-(4-methoxyphenyl)penta-2,4-dienal, 5-(3,4-dimethoxyphenyl)penta-2,4-dienal, 5-(2,4-dimethoxyphenyl)penta-2,4-dienal, 5-(4-piperidinophenyl)penta-2,4-dienal, 5-(4-morpholinophenyl)penta-2,4-dienal, 5-(4-pyrrolidinophenyl)penta-2,4-dienal, 6-(4-dimethylaminophenyl)hexa-3,5-dien-2-one, 6-(4-diethylaminophenyl)hexa-3,5-dien-2-one, 6-(4-methoxyphenyl)hexa-3,5-dien-2-one, 6-(3,4-dimethoxyphenyl)hexa-3,5-dien-2-one, 6-(2,4-dimethoxyphenyl)hexa-3,5-dien-2-one, 6-(4-piperidinophenyl)hexa-3,5-dien-2-one, 6-(4-morpholinophenyl)hexa-3,5-dien-2-one, 6-(4-pyrrolidinophenyl)hexa-3,5-dien-2-one, 5-(4-dimethylamino-1-naphthyl)penta-3,5-dienal, 9-methyl-3-carbazole aldehyde, 9-ethyl-3-carbazole aldehyde, 3-acetylcarbazole, 3,6-diacetyl-9-ethylcarbazole, 3-acetyl-9-methylcarbazole, 1,4-dimethyl-3-carbazole aldehyde, 1,4,9-trimethyl-3-carbazole aldehyde, 4-formyl-1-methylpyridinium-, 2-formyl-1-methylpyridinium-, 4-formyl-1-ethylpyridinium-, 2-formyl-1-ethylpyridinium-, 4-formyl-1-benzylpyridinium-, 2-formyl-1-benzylpyridinium-,4-formyl-1,2-dimethylpyridinium-, 4-formyl-1,3-dimethylpyridinium-, 4-formyl-1-methylquinolinium-, 2-formyl-1-methylquinolinium-, 4-acetyl-1-methylpyridinium-, 2-acetyl-1-methylpyridinium-, 4-acetyl-1-methylquinolinium-, 5-formyl-1-methylquinolinium-, 6-formyl-1-methylquinolinium-, 7-formyl-1-methylquinolinium-, 8-formyl-1-methylquinolinium, 5-formyl-1-ethylquinolinium-, 6-formyl-1-ethylquinolinium-, 7-formyl-1-ethylquinolinium-, 8-formyl-1-ethylquinolinium, 5-formyl-1-benzylquinolinium-, 6-formyl-1-benzylquinolinium-, 7-formyl-1-benzylquinolinium-, 8-formyl-1-benzylquinolinium, 5-formyl-1-allylquinolinium-, 6-formyl-1-allylquinolinium-, 7-formyl-1-allylquinolinium- and 8-formyl-1-allylquinolinium-, 5-acetyl-1-methylquinolinium-, 6-acetyl-1-methylquinolinium-, 7-acetyl-1-methylquinolinium-, 8-acetyl-1-methylquinolinium, 5-acetyl-1-ethylquinolinium-, 6-acetyl-1-ethylquinolinium-, 7-acetyl-1-ethylquinolinium-, 8-acetyl-1-ethylquinolinium, 5-acetyl-1-benzylquinolinium-, 6-acetyl-1-benzylquinolinium-, 7-acetyl-1-benzylquinolinium-, 8-acetyl-1-benzylquinolinium, 5-acetyl-1-allylquinolinium-6-acetyl-1-allylquinolinium-, 7-acetyl-1-allylquinolinium- and 8-acetyl-1-allylquinolinium-, 9-formyl-10-methylacridinium-, 4-(2′-formylvinyl)-1-methylpyridinium-, 1,3-dimethyl-2-(4′-formylphenyl)-benzimidazolium-, 1,3-dimethyl-2-(4′-formylphenyl)-imidazolium-, 2-(4′-formylphenyl)-3-methylbenzothiazolium-, 2-(4′-acetylphenyl)-3-methylbenzothiazolium-, 2-(4′-formylphenyl)-3-methylbenzoxazolium-, 2-(5′-formyl-2′-furyl)-3-methylbenzothiazolium-, 2-(5′-formyl-2′-furyl)-3-methylbenzothiazolium-, 2-(5′-formyl-2′-thienyl)-3-methylbenzothiazolium-, 2-(3′-formylphenyl)-3-methylbenzothiazolium-, 2-(4′-formyl-1-naphthyl)-3-methylbenzothiazolium-, 5-chloro-2-(4′-formylphenyl)-3-methylbenzothiazolium-, 2-(4′-formylphenyl)-3,5-dimethylbenzothiazolium-benzenesulfonate, -p-toluenesulfonate, -methanesulfonate, -perchlorate, -sulfate, -chloride, -bromide, -iodide, -tetrachlorozincate, -methylsulfate, trifluormethanesulfonate, -tetrafluoroborate, isatin, 1-methyl-isatin, 1-allyl-isatin, 1-hydroxymethyl-isatin, 5-chloro-isatin, 5-methoxy-isatin, 5-nitro-isatin, 6-nitro-isatin, 5-sulfo-isatin, 5-carboxy-isatin, quinisatin, 1-methylquinisatin, as well as any mixtures of the above compounds.

31. Agent according to claim 1, wherein the compounds of the component A, the compounds of the component B, and optionally the compounds of the components C and D are each comprised in an amount of 0.03 to 65 mmol, in particular from 1 to 40 mmol, based on 100 g of the total dye.

32. Agent according to claim 1, additionally comprising at least one substantive dye, preferably in a quantity of 0.01 to 20 wt. %, based on the total dye.

33. Agent according to claim 1, additionally comprising anionic, zwitterionic or non-ionic surfactants.

34. Use of at least one agent according to claim 1, as a coloration component in hair dyes.

35. Method for dyeing keratin-containing fibers, especially human hair, in which an agent according to claim 1, together with conventional cosmetic ingredients, is applied onto the keratin-containing fibers, left on the fibers for some time, usually about 15-30 minutes, and then rinsed out again or washed out with a shampoo.
